SEQUENCE LISTING (15 sequences)

SEQ ID NO: 1 - DNA, 39 bp, Homo sapiens
tcgatccctcactcaccccccgcgtctagggagggtccg

SEQ ID NO: 2 - DNA, 23 bp, Homo sapiens
tgattataggyttagggagggtg

SEQ ID NO: 3 - DNA, 23 bp, Artificial
  Synthetic DNA
aactaaaaaataaataaaaaaca

SEQ ID NO: 4 - DNA, 18 bp, Artificial
  Synthetic DNA
aacaaatccctcccaaac

SEQ ID NO: 5 - DNA, 17 bp, Artificial
  Synthetic DNA
gtttgggagggatttgt

SEQ ID NO: 6 - DNA, 24 bp, Artificial
  Synthetic DNA
ctgttttttatttattttttagtt

SEQ ID NO: 7 - DNA, 22 bp, Artificial
  Synthetic DNA
aactaaaaaataaataaaaaac

SEQ ID NO: 8 - DNA, 18 bp, Artificial
  Synthetic DNA
agcaaatccctcccaaac

SEQ ID NO: 9 - DNA, 17 bp, Artificial
  Synthetic DNA
gtttgggagggatttgc

SEQ ID NO: 10 - DNA, 24 bp, Artificial
  Synthetic DNA
ccgttttttatttattttttagtt

SEQ ID NO: 11 - DNA, 36 bp, Homo sapiens
ccagagggtggggcggaccgagtgcgctcggcggct

SEQ ID NO: 12 - DNA, 18 bp, Artificial
  Synthetic DNA
aaccnccnaacncactca

SEQ ID NO: 13 - DNA, 27 bp, Artificial
  Synthetic DNA
gcagattgaatccnccccaccctctaa

SEQ ID NO: 14 - DNA, 18 bp, Artificial
  Synthetic DNA
aaccnccnaacncactcg

SEQ ID NO: 15 - DNA, 27 bp, Artificial
  Synthetic DNA
tctcaccggatccnccccaccctctaa

(19) United States
(12) Patent Application Publication
(10) Pub. No.: US 2006/0234252 A1
(43) Pub. Date: Oct. 19, 2006

(54) Methods and kits for methylation detection

(75) Inventor: Mark R. Andersen, Carlsbad, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/173,569

(22) Filed: Jul. 1, 2005

Related U.S. Application Data
(60) Provisional application No. 60/585,131, filed on Jul. 2, 2004.

(51) Int. Cl. C12Q 1/68

(52) U.S. Cl. 435/6

(57) ABSTRACT

Methods for determining the methylation state of at least one target nucleotide that employ a reaction catalyzed by a structure-specific nuclease, typically coupled with a ligation reaction are disclosed. By detecting the cleaved flap, a ligation product, a ligation product surrogate, a hybridization complex, or combinations thereof, one can infer the degree to which the corresponding target nucleotide is methylated. Certain of the disclosed methods are particularly useful for evaluating bisulfite-treated target sequences and determining the degree of target nucleotide methylation. The disclosed methods are well suited for rapidly analyzing a large number of target sequences, typically in one or more multiplex reactions. Kits for performing coupled nuclease and ligase methylation detection assays are also disclosed.

### CROSS REFERENCE TO RELATED APPLICATIONS

This application claims priority benefit under 35 U.S.C. § 119(e) from U.S. Provisional Patent Application No. 60/585,131, filed Jul. 2, 2004, which is incorporated herein by reference.

### FIELD

The present teachings generally relate to methods and kits for determining the methylation state of a target nucleotide. More specifically, the disclosed methods and kits employ at least one nuclease cleavage reaction, typically coupled with at least one ligation reaction, to generate a detectable signal that can be used to determine the degree of target nucleotide methylation.

### BACKGROUND

The methylation of cytosine residues in DNA is an important epigenetic alteration in eukaryotes. In humans and other mammals methylcytosine is found almost exclusively in cytosine-guanine (CpG) dinucleotides. DNA methylation plays an important role in gene regulation and changes in methylation patterns are reportedly involved in human cancers and certain human diseases. Among the earliest and most common genetic alterations observed in human malignancies is the aberrant methylation of CpG islands, particularly CpG islands located within the 5′ regulatory regions of genes, causing alterations in the expression of such genes. Subsequently, there is great interest in using DNA methylation markers as diagnostic indicators for early detection, risk assessment, therapeutic evaluation, recurrence monitoring, and the like (see, Widschwendter et al., Clin. Cancer Res. 10:565-71, 2004; Dulaimi et al., Clin. Cancer Res. 10:1887-93, 2004; Topaloglu et al., Clin. Cancer Res. 10:2284-88, 2004; Laird, Nature Reviews, 3:253-266, 2003; Fraga et al., BioTechniques 33:632-49, 2002; Adorjan et al., Nucleic Acids Res. 30(5):e21, 2002; and Colella et al., BioTechniques, 35(1):146-150, 2003). There is also great scientific interest in DNA methylation for studying embryogenesis, cellular differentiation, transgene expression, transcriptional regulation, and maintenance methylation, among other things. In lower organisms such as bacteria, adenine rather than cytosine is typically methylated, i.e., N⁶-methlyadenosine.

### SUMMARY

The present teachings are directed to methods and kits for determining the degree of methylation of a specific nucleotide, generally but not exclusively cytosine residues, in a polynucleotide sequence. In certain embodiments, target sequences are converted, prior to probe binding, while in other embodiments, the target sequences are not converted. According to certain disclosed methods, a multiplicity of different target nucleotides are interrogated using a first cleavage probe set for each target nucleotide and a multiplicity of first ligation products are generated. In certain embodiments, a target sequence is converted (modified) and a cleavage probe comprises a universal base. In certain embodiments, a target sequence is converted and a multiplicity of different cleavage probes designed to interrogate the same target nucleotide comprise degenerate bases.

According to certain disclosed methods, target sequences are combined with a first cleavage probe set, comprising a first (upstream) cleavage probe and a second (downstream) cleavage probe, a cleaving enzyme, and a ligation agent to form a cleavage-ligation reaction composition. The upstream cleavage probe comprises a first target region-binding portion and the downstream cleavage probe comprises a second target region-binding portion that are complementary with the first region and the second region of the target sequence, respectively. The 3′-end of the upstream probe first target region-binding portion and the 5′-end of the downstream probe second target region-binding portion overlap by a nucleotide. In certain embodiments, this overlap or “flap” portion of the second cleavage probe comprises at least two nucleotides. Under appropriate conditions, the first and the second cleavage probes anneal with the first and second regions of the target sequence, respectively, to form a first hybridization complex. In certain embodiments, the second cleavage probe does not initially include a portion that overlaps the 3′-end of the first cleavage probe, but it is “created” by extending the 3′-end of the adjacently hybridized first cleavage probe, thereby forming a first hybridization complex. The cleavage-ligation reaction composition is subjected to a cleavage-ligation cycle as follows. The flap portion of the second cleavage probe is cleaved under appropriate reaction conditions, releasing the cleaved flap and in so doing, forming a second hybridization complex comprising the target sequence, the first cleavage probe and a fragment of the second cleavage probe adjacently hybridized to the first cleavage probe. Provided that the adjacently hybridized first probe and second probe fragment are suitable for ligation, they are ligated together by the ligation agent to generate a first ligation product and in so doing, form a third hybridization complex comprising the target sequence and the first ligation product.

Denaturation of the third hybridization complex releases the first ligation product from the corresponding target sequence. In certain embodiments, a cleaved flap or a first ligation product is detected. The target sequence and the first ligation product can also serve as templates for further reactions. The target sequence can hybridize with additional first probes and additional second probes of the first cleavage probe set to generate additional first ligation products and first cleaved flaps. The first ligation product, when combined with a second cleavage probe set, can form a fourth hybridization complex, comprising the first ligation product, and a first and a second probe of the second cleavage probe set. Under appropriate reaction conditions, a second cleaved flap is generated by a cleaving enzyme and a fifth hybridization complex is formed, comprising the first ligation product, the first cleavage probe of the second cleavage probe set and adjacently hybridized, and a fragment of the downstream probe of the second cleavage probe set. Provided that the adjacently hybridized first probe and the second probe fragment are suitable for ligation, they can be ligated together by a ligation agent to generate a second ligation product and in so doing, form a sixth hybridization complex, comprising the first and second ligation products. Denaturation of the sixth hybridization complex releases the first and second ligation products.

In certain embodiments, the first ligation product, the second ligation product, the target sequence, or combinations thereof, are combined with appropriate cleavage probe sets and cycled through additional coupled cleavage-ligation reactions to generate additional ligation products and additional cleaved flaps. In certain embodiments, the first ligation product is combined with a first ligation probe set and a ligation agent to form a first ligation reaction composition. Under appropriate reaction conditions, the first and second ligation probes hybridize with the corresponding first ligation product to form a seventh hybridization complex. Provided that the two adjacently hybridized probes are suitable for ligation, they are ligated together by a ligation agent to generate a third ligation product and form an eighth hybridization complex comprising the first and the third ligation products. Likewise, the second ligation product can be combined with a second ligation probe set to form a ninth hybridization complex and a fourth ligation product can be generated by a ligation agent and a tenth hybridization complex is formed comprising the second and the fourth ligation products. In certain embodiments, the third ligation product or the fourth ligation product are detected.

In certain embodiments, a cleavage probe, a ligation probe, a cleaved flap, a ligation product, an amplified ligation product, a digested ligation product, or combinations thereof, comprise a reporter group, a hybridization tag, a mobility modifier, a reporter probe-binding portion, a primer-binding portion, an affinity tag, or combinations thereof. In certain embodiments, the 5′-end of cleavage probe does not comprise a nucleotide 5′-phosphate group. In certain embodiments, the 3′-end of a cleavage probe does not comprise a nucleotide 3′-hydroxyl group. In certain embodiments, a second cleavage probe comprises at least two different reporter groups, including without limitation, a fluorescent reporter group and a quencher. In certain embodiments, a target sequence is treated with a modifying agent, including without limitation, sodium bisulfite, to convert a nucleotide (i.e., the target sequence is modified). In certain embodiments, multiplex methods are disclosed in which: a multiplicity of different target sequences are each interrogated with a corresponding cleavage probe set in the same assay or in a parallel assay; a multiplicity of different cleaved flaps and a multiplicity of different ligation products are generated; and a cleaved flap, a ligation product, or a cleaved flap and a ligation product, are detected and the degree of methylation for a multiplicity of corresponding target nucleotides is determined.

In some methods for determining the degree of target nucleotide methylation, a target sequence is reacted with a first cleavage probe set under effective conditions for the first cleavage probe and the second cleavage probe to anneal to the first target region and the second target region, respectively, to form a first hybridization complex. A cleaving enzyme cleaves the flap portion of the second cleavage probe of the first hybridization complex to generate a cleaved flap and form a second hybridization complex, comprising a first cleavage probe, a hybridized fragment of the second cleavage probe having a 5′ end comprising a nucleotide 5-phosphate group adjacent to the 3′-end of the hybridized first cleavage probe. In the presence of a ligation agent and under appropriate reaction conditions, the first cleavage probe and the hybridized fragment of the second cleavage probe are ligated together (provided that they are suitable for ligation) to generate a first ligation product and form a third hybridization complex comprising the target sequence and the first ligation product. The first hybridization complex is denatured to release the target sequence and the first ligation product. When the cycle of the reacting step, the cleaving step, and the ligating step are repeated, a plurality of third hybridization complexes are generated and upon denaturation, a plurality of first ligation products are released.

In certain embodiments, methods for determining the degree of target nucleotide methylation are disclosed comprising: a step for interrogating a target nucleotide; a step for generating a cleaved flap; a step for generating a first ligation product; and a step for determining the degree of methylation of a target nucleotide. In certain embodiments, such methods further comprise: a step for generating a second cleaved flap; a step for generating a second ligation product; a step for generating an amplified ligation product; a step for generating a digested ligation product; a step for gap-filling; a step for extending a first cleavage probe; or combinations thereof. In certain embodiments, such methods are multiplexed, automated, semi-automated, or combinations thereof. Those skilled in the art will appreciate that the step for interrogating can be performed using the first cleavage probe sets disclosed herein; that the step for generating a first cleaved flap or a second cleaved flap can be performed using the cleaving enzymes disclosed herein; that the step for generating a first ligation product, a second ligation product, a third ligation product, a fourth ligation product, or combinations thereof, can be performed using the ligation agents or ligation techniques disclosed herein; that the step for generating an amplified ligation product, the step for gap-filling, and step for extending a first cleavage probe can be performed using the amplification means disclosed herein; that the step for generating a digested ligation product can be performed using the digesting means disclosed herein; and that the step for determining the degree of methylation of a target nucleotide can be performed using the determining means disclosed herein.

Each of the ligation probe sets comprise a first ligation probe comprising a first ligation product-binding portion and a second ligation probe comprising a second ligation product-binding portion. In certain embodiments, the first and second probes of a ligation probe set are designed to adjacently hybridize on the corresponding ligation product such that the 3′-end of the first ligation probe and the 5′-end of the second ligation probe are immediately adjacent to each other. In certain embodiments, the first and corresponding second probe of at least one probe set do not adjacently hybridize initially, but the 3′-end of the first probe is extended by a gap-filling step until it becomes adjacent to the 5′-end of the corresponding second probe, for example but not limited to gap LCR and other gap-filling techniques (see, e.g., Osiowy, J. Clin. Micro. 40:2566-71, 2002; and Abravaya et al., Nucl. Acids Res. 23:675-82, 1995).

The ligation agents of the current teachings include a wide variety of enzymatic and chemical reagents and techniques, including without limitation, autoligation and photoligation. Thus, in certain embodiments, the 3′-end of a first cleavage probe, an upstream ligation probe, or a first cleavage probe and an upstream ligation probe, terminates in a nucleotide 3′-hydroxyl group and the 5-end of the corresponding downstream probe terminates in a nucleotide 5′-phosphate group. In other embodiments, the 3′-end of a first cleavage probe or an upstream ligation probe terminates in a group other than a nucleotide 3′-hydroxyl group and the 5-end of the corresponding downstream probe terminates in a group other than a nucleotide 5′-phosphate group.

The disclosed methods and kits typically comprise a ligation agent. In certain embodiments, the ligation agent comprises a ligase, such as DNA ligase or RNA ligase, including without limitation, the bacteriophage T4 (T4) DNA ligase, T4 RNA ligase, E. coli DNA ligase, or E. coli RNA ligase. In certain embodiments a ligase comprises a thermostable ligase. Exemplary thermostable ligases include without limitation, Thermus species ligases, for example but not limited to Thermus species AK16D ligase, Pfu ligase, Afu ligase, and the like, including ligases of bacteriophages that infect thermophilic or hyperthermophilic eubacteria and viruses that infect archaea, formerly known as archaebacteria.

In certain embodiments, ligation is performed non-enzymatically. While not limiting, non-enzymatic ligation typically includes both photoligation and chemical ligation, such as, autoligation and ligation in the presence of an “activating” or reducing agent. Non-enzymatic ligation can utilize specific reactive groups on the respective 3′ and 5′ ends of the probes to be ligated. Thus, in certain embodiments of the disclosed methods and kits, the ligation agent comprises an “activating” or reducing agent. In certain embodiments, the ligation agent comprises a photoligation source. In certain embodiments, probes are provided that comprise reactive groups that are suitable for non-enzymatic ligation. Thus, the disclosed ligation means comprise a wide range of enzymatic, chemical and photochemical techniques and reagents for joining the ends of suitable probes.

Kits for performing the disclosed methods are also provided. In certain embodiments, kits comprise a cleavage probe set, a ligation probe set, a primer, a hybridization tag, a hybridization tag complement, a mobility modifier, a reporter group, an affinity tag, a reporter probe, or combinations thereof. In certain embodiments, kits comprise: a cleaving enzyme, for example but not limited to, a structure-specific nuclease, including without limitation, a flap endonuclease, a bacterial polymerase comprising 5′-3′ exonuclease activity, and the 5′-3′ exonuclease domain of such a bacterial polymerase; a ligation agent; a polymerase; a digesting agent, including without limitation, a nuclease, a restriction enzyme, and a chemical digestion means; a Substrate; or combinations thereof. In certain embodiments, kits are disclosed that comprise a cleaving means, a ligating means, an amplifying means, a separating means, a digesting means, a detecting means, an analyzing means, or combinations thereof. In certain embodiments, kits further comprise a modifying means, for example but not limited to, sodium bisulfite.

These and other features of the present teachings are set forth herein.

### BRIEF DESCRIPTION OF THE DRAWINGS

The following schematic drawings are for illustration purposes only and depict certain exemplary embodiments of the current teachings. The schematics are not necessarily drawn to scale and they should not be construed as limiting the current teachings in any way.

FIGS. 1A-B: schematically depicts illustrative embodiments of certain coupled cleavage-ligation reactions using bisulfite-treated DNA (“converted target”). A cleavage probe set comprising two related probe pairs is shown in FIG. 1A. The first probe pair (for illustration purposes, the “A” probe pair) comprises a first cleavage probe, “1A” comprising a first target region-binding portion, and a second cleavage probe “2A” comprising a 5′ flap portion upstream from a second target region-binding portion and a biotin affinity tag (“b”), wherein each probe in this probe pair comprises an “A” pivotal complement. The second probe pair (for illustration purposes, the “G” probe pair) comprises a first cleavage probe, “1 G” comprising a first target region-binding portion and a second cleavage probe “2G” comprising a 5′ flap portion upstream from a second target region-binding portion and a digoxigenin affinity tag (“DIG”), wherein each probe in this probe pair comprises a “G” pivotal complement. This exemplary cleavage probe set is reacted with a converted target sequence, comprising a first target region and a second target region (“first region” and “second region”, respectively) and an initially undetermined target nucleotide (“?”; located in the overlap of the first and the second target regions), to form a first hybridization complex. The hybridized second cleavage probe is cleaved by a cleaving enzyme to form a second hybridization complex and release a cleaved flap comprising the pivotal complement from the second cleavage probe and additional upstream flap sequences (“cleaved flap”). The probes of the second hybridization complex are ligated together by a ligation agent to generate a first ligation product (“1 LP”) and in so doing, form a third hybridization complex. FIG. 1B schematically depicts a fourth hybridization complex, comprising a second cleavage probe set, wherein the second cleavage probe comprises a dinitrophenol affinity tag (“DNP”); a fifth hybridization complex; and a sixth hybridization complex, comprising the 1LP and the second ligation product (“2LP”) each comprising an affinity tag. 1G: first cleavage probe of the first cleavage probe pair comprising a “G” nucleotide pivotal complement; 2G: second cleavage probe of the first cleavage probe pair comprising a G nucleotide as the pivotal complement and a flap sequence upstream from the second target region-binding portion; 2A*: fragment on the 2A second cleavage probe after cleavage of the flap; 1 LP: first ligation product; 2LP: second ligation product; and “U”: converted target nucleotide.

FIG. 2: schematically depicts another exemplary embodiment of two parallel reactions comprising converted target sequences. One coupled cleavage-ligation reaction (shown as “U-specific assay”) comprises a first cleavage probe set comprising a first cleavage probe with an A nucleotide as the pivotal complement (“1A”) and a second cleavage probe with an A nucleotide as the pivotal complement and an upstream flap portion (“2A”). The second coupled cleavage-ligation reaction (shown as “C-specific assay”) comprises a first cleavage probe set comprising a probe pair wherein the 3′-end of the first cleavage probe comprises a G nucleotide as the pivotal complement (“1G”) and the second cleavage probe comprises a G nucleotide as the pivotal complement (“2G”). U: uracil, i.e., a converted target nucleotide; mC: 5-methylcytosine, i.e, the target nucleotide was not converted; 2G*: hybridized fragment of the second cleavage probe; 1 LP-A: first ligation product of the 1A:2A probe pair; 1 LP-G: first ligation product of the 1 G:2G probe pair.

FIG. 3: schematically depicts another exemplary embodiment comprising two parallel cleavage-ligation reactions where the target sequences in one reaction have been bisulfite treated to convert “C” target nucleotides (shown as Tube 1: “converted” targets) and the target sequences in the second cleavage-ligation reaction have not been bisulfite treated (shown as Tube 2: “native” targets).

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. For example but not limited to, “a probe” means that more than one probe can be present; for example but not limited to, one or more copies of a particular probe species, as well as one or more versions of a particular probe type. The use of “or” means “and/or” unless otherwise apparent from the context. Also, the use of “comprise”, “comprises”, “comprising”, “include”, “includes”, and “including” are not intended to be limiting.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. In the event that an incorporated literature and similar material differs from or contradicts this application, including but not limited to defined terms, term usage, described techniques, or the like, this application controls.

I. Definitions

The term “affinity tag” as used herein refers to a component of a multi-component complex, wherein the components of the multi-component complex specifically interact with or bind to each other. Exemplary multiple-component affinity tag complexes include without limitation, ligands and their receptors, for example but not limited to, avidin-biotin, streptavidin-biotin, and derivatives of biotin, streptavidin or avidin, including without limitation, 2-iminobiotin, desthiobiotin, NeutrAvidin (Molecular Probes, Eugene, Oreg.), CaptAvidin (Molecular Probes), and the like; binding proteins/peptides, including without limitation, maltose-maltose binding protein (MBP), calcium-calcium binding protein/peptide (CBP); epitope tags, for example but not limited to c-MYC (e.g., EQKLISEEDL), HA (e.g., YPYDVPDYA), VSV-G (e.g., YTDIEMNRLGK), HSV (e.g., QPELAPEDPED), V5 (e.g., GKPIPNPLLGLDST), and FLAG Tag™ (e.g., DYKDDDDKG), and their corresponding anti-epitope antibodies; haptens, for example but not limited to dinitrophenol (“DNP”) and digoxigenin (“DIG”), and their corresponding antibodies; aptamers and their corresponding targets; poly-His tags (e.g., penta-His and hexa-His) and their binding partners, including without limitation, corresponding metal ion affinity chromatography (IMAC) materials and anti-poly-His antibodies; fluorophores and their corresponding anti-fluorophore antibodies; and the like. In certain embodiments, affinity tags are part of a separating means or part of a detecting means.

The term “cleaving enzyme” refers to any enzyme, including enzymatically active mutants or variants thereof, that can, when combined with: a first hybridization complex and under appropriate conditions, generate a first cleaved flap to form a second hybridization complex; or a third hybridization complex and under appropriate conditions, generate a second cleaved flap to form a fourth hybridization complex. Such cleaving enzymes include without limitation, structure-specific nucleases, for example but not limited to, certain DNA polymerases from bacteria and bacteriophages, including isolated 5′exonuclease domains thereof; eukaryotic flap endonucleases; and archaeal flap endonucleases (see, e.g., Lyamichev et al., Science 260:778-83; Li et al., J. Biol. Chem. 270:22109-12, 1995; Wu et al., Nucl. Acids Res. 24:2036-43, 1996; Hosfield et al., J. Biol. Chem. 273:27154-61, 1998; Kaiser et al., J. Biol. Chem. 274:21387-94, 1999).

The term “or combinations thereof” as used herein refers to all permutations and combinations of the listed items preceding the term. For example, “A, B, C, or combinations thereof” is intended to include: A, B, C, AB, AC, BC, or ABC, and if order is important in a particular context, also BA, CA, CB, CBA, BCA, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term refers. For example, a first probe of a particular probe pair corresponds to a second probe of the same probe pair, and vice versa. At least one primer is designed to anneal with the primer-binding portion of a corresponding probe, a corresponding ligation product, a corresponding amplified ligation product, a corresponding digested ligation product, a corresponding digested amplified ligation product, or combinations thereof. The sequence-specific portions of the probes of a particular probe set are designed to hybridize with a complementary or substantially complementary region of the corresponding target sequence or the corresponding ligation product. A particular affinity tag binds to the corresponding affinity tag; a particular hybridization tag anneals with its corresponding hybridization tag complement; and so forth.

The term “enzymatically active mutants or variants thereof” when used in reference to one or more enzyme, such as a cleaving enzyme, a polymerase, a ligase, a nuclease, or the like, refers to a polypeptide derived from the corresponding enzyme that retains at least some of the desired enzymatic activity, such as cleaving, ligating, amplifying, or digesting, as appropriate. An enzymatically active mutant or variant differs from the “generally-accepted” or consensus sequence for that enzyme by an amino acid, including, but not limited to, substitutions of an amino acid, addition of an amino acid, deletion of an amino acid, and modifications to the amino acids themselves. Provided that the resulting mutant or variant retains at least some catalytic activity. “Amino acid” as used herein refers to any amino acid, natural or non-natural, that may be incorporated, either enzymatically or synthetically, into a polypeptide or protein.

Also within the scope of this term are: enzymatically active fragments, including without limitation, cleavage products, for example but not limited to, Klenow fragment, Stoffel fragment, and recombinantly-expressed fragments or polypeptides that are smaller in size than the corresponding enzyme; mutant forms of the corresponding enzyme, including but not limited to, naturally-occurring mutants, mutants that are generated using physical or chemical mutagens, and genetically engineered mutants, for example but not limited to random and site-directed mutagenesis techniques; amino acid insertions and deletions, and changes due to nucleic acid nonsense mutations, missense mutations, and frameshift mutations (see, e.g., Sriskanda and Shuman, Nucl. Acids Res. 26(2):525-31, 1998; Odell et al., Nucl. Acids Res. 31(17):5090-5100, 2003); reversibly modified nucleases, ligases, and polymerases, for example but not limited to those described in U.S. Pat. No. 5,773,258; biologically active polypeptides obtained from gene shuffling techniques (see, e.g., U.S. Pat. Nos. 6,319,714 and 6,159,688), splice variants, both naturally occurring and genetically engineered; polypeptides corresponding at least in part to an enzyme that comprise modifications to an amino acid of the native sequence, including without limitation, adding, removing or altering glycosylation, disulfide bonds, hydroxyl side chains, and phosphate side chains, methylation, alkylation, biotinylation, or crosslinking, provided such modified polypeptides retain at least some of the desired catalytic activity; and the like. Expressly within the meaning of the term “enzymatically active mutants or variants thereof” when used in reference to a particular enzyme are enzymatically active mutants of that enzyme, enzymatically active variants of that enzyme, or enzymatically active mutants of that enzyme and enzymatically active variants of that enzyme.

The skilled artisan will readily be able to measure enzymatic activity using an appropriate assay known in the art. Thus, an appropriate assay for polymerase catalytic activity might include, for example, measuring the ability of a variant to incorporate, under appropriate conditions, rNTPs or dNTPs into a nascent polynucleotide strand in a template-dependent manner. Likewise, an appropriate assay for ligase catalytic activity might include, for example, the ability to ligate adjacently hybridized oligonucleotides comprising appropriate reactive groups, such as disclosed herein. Protocols for such assays may be found in, among other places, Molecular Cloning, A Laboratory Manual, Cold Spring Harbor Press, 3d ed., 2001 (“Sambrook and Russell”); Sambrook, Fritsch, and Maniatis, Molecular Cloning, A Laboratory Manual, Cold Spring Harbor Press, 2d ed., 1989 (“Sambrook et al.”); Ausubel et al., Current Protocols in Molecular Biology, John Wiley & Sons, Inc. (including supplements through June 2004)(“Ausubel et al.”); and Housby and Southern, Nucl. Acids Res. 26:4259-66, 1998).

The terms “groove binder” and “minor groove binder” refer to small molecules that fit into the minor groove of double-stranded DNA, typically in a sequence specific manner. Generally, minor groove binders are long, flat molecules that can adopt a crescent-like shape and thus, fit snugly into the minor groove of a double helix, often displacing water. Minor groove binding molecules typically comprise several aromatic rings connected by bonds with torsional freedom, such as but not limited to, furan, benzene, or pyrrole rings. Exemplary minor groove binders include without limitation, antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues. In certain embodiments, a minor groove binder is a component of a cleavage probe, a ligation probe, a primer, a reporter probe, a hybridization tag complement, or combinations thereof. Detailed descriptions of minor groove binders can be found in, among other places, Nucleic Acids in Chemistry and Biology, 2d ed., Blackburn and Gait, eds., Oxford University Press, 1996 (“Blackburn and Gait”), particularly in section 8.3; Kumar et al., Nucl. Acids Res. 26:831-38, 1998; Kutyavin et al., Nucl. Acids Res. 28:655-61, 2000; Turner and Denny, Curr. Drug Targets 1:1-14, 2000; Kutyavin et al., Nucl. Acids Res. 25:3718-25, 1997; Lukhtanov et al., Bioconjug. Chem. 7:564-7, 1996; Lukhtanov et al., Bioconjug. Chem. 6: 418-26, 1995; U.S. Pat. No. 6,426,408; and PCT Published Application No. WO 03/078450. Primers and reporter probes comprising minor groove binders are commercially available from, among other places, Applied Biosystems (Foster City, Calif.) and Epoch Biosciences (Bothell, Wash.).

The terms “hybridizing” and “annealing”, and their grammatical equivalents (meaning variations of these terms such as annealed, hybridization, anneal, hybridizes, and so forth), are used interchangeably and mean the base-pairing interaction of one nucleic acid with another nucleic acid that results in the formation of a duplex, triplex, or other higher-ordered structure. The primary interaction is typically base specific, e.g., A:T, A:U and G:C, by Watson-Crick and Hoogsteen-type hydrogen bonding. In certain embodiments, base-stacking and hydrophobic interactions may also contribute to duplex stability. Conditions under which nucleic acid probes and primers hybridize to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the probes and the complementary sequences, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by persons of ordinary skill in the art, without undue experimentation. The term “specifically hybridize” means that the two probes bind a target or a ligation product with sufficient specificity to differentiate it from a non-target molecule or non-corresponding ligation product, as appropriate.

The term “hybridization tag” as used herein refers to an oligonucleotide sequence that can be used for: separating the element (e.g., ligation products, surrogates, ZipChute™ reagents, etc.) of which it is a component or to which it is bound, including without limitation, bulk separation; tethering or attaching the element to which it is bound to a Substrate, which may or may not include separating; annealing a corresponding hybridization tag complement; or combinations thereof. In certain embodiments, the same hybridization tag is used with a multiplicity of different elements to effect bulk separation, Substrate attachment, or combinations thereof. A “hybridization tag complement” typically refers to an oligonucleotide that comprises a sequence of nucleotides that are complementary to and specifically hybridize with at least part of the corresponding hybridization tag. In various embodiments, hybridization tag complements serve as capture moieties for attaching a hybridization tag:element complex to a Substrate; serve as “pull-out” sequences for bulk separation procedures; or both as capture moieties and as pull-out sequences. In certain embodiments, a hybridization tag complement comprises a reporter group, a mobility modifier, a reporter probe-binding portion, or combinations thereof. In certain embodiments, a hybridization tag complement is annealed to a corresponding hybridization tag and, subsequently, at least part of that hybridization tag complement is released and detected. In certain embodiments, determining comprises detecting one or more reporter groups on or attached to a hybridization tag complement or at least part of a hybridization tag complement.

Typically, hybridization tags and their corresponding hybridization tag complements are selected to minimize: internal self-hybridization; cross-hybridization with different hybridization tag species, nucleotide sequences in a reaction composition, including but not limited to gDNA, different species of hybridization tag complements, target-specific portions of probes, primers, and the like; but should be amenable to facile hybridization between the hybridization tag and its corresponding hybridization tag complement. Hybridization tag sequences and hybridization tag complement sequences can be selected by any suitable method, for example but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65, 1998). Descriptions of hybridization tags can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein); and Gerry et al., J. Mol. Biol. 292:251-262, 1999) (referred to as “zip-codes” and “zip-code complements” therein). Those in the art will appreciate that a hybridization tag and its corresponding hybridization tag complement are, by definition, complementary to each other and that the terms hybridization tag and hybridization tag complement are relative and can essentially be used interchangeably in most contexts.

Hybridization tags can be located at or near the end of a probe, a primer, a ligation product, a ligation product surrogate, or combinations thereof; or they can be located internally. In certain embodiments, a hybridization tag is attached to a probe, a primer, a ligation product, a ligation product surrogate, or combinations thereof, via a linker arm. In certain embodiments, the linker arm is cleavable.

In certain embodiments, hybridization tags are at least 12 bases in length, at least 15 bases in length, 12-60 bases in length, or 15-30 bases in length. In certain embodiments, a hybridization tag is 12, 15, 20, 21, 22, 23, 24, 25, 26, 27, 28, 29, 30, 45, or 60 bases in length. In certain embodiments, at least two hybridization tag:hybridization tag complement duplexes have melting temperatures that fall within a ΔTₘ range (Tₘₐₓ-Tₘᵢₙ) of no more than 10° C. of each other. In certain embodiments, at least two hybridization tag:hybridization tag complement duplexes have melting temperatures that fall within a ΔTₘ range of 5° C. or less of each other.

A “ligation agent” according to the present invention comprises any number of enzymatic or non-enzymatic agents that can effect ligation of nucleic acids to one another, including without limitation, ligases, chemical ligation agents and photoligation. For example, ligase is an enzymatic ligation agent that, under appropriate conditions, forms phosphodiester bonds between the 3′-OH and the 5′-phosphate of adjacent probes. Temperature sensitive ligases, include, but are not limited to, bacteriophage T4 ligase and E. coli ligase. Exemplary thermostable ligases include, without limitation, Afu ligase, Taq ligase, Tfl ligase, Mth ligase, Tth ligase, Tth HB8 ligase, Tsc ligase, Thermus species AK16D ligase, Ape ligase, Lig_{Tk} ligase, Aae ligase, Rm ligase, and Pfu ligase (see, e.g., Housby et al., Nucl. Acids Res. 28:e10, 2000; Tong et al., Nucl. Acids Res. 28:1447-54, 2000; Nakatani et al., Eur, J. Biochem. 269:650-56, 2002; and Sriskanda et al., Nucl. Acids Res. 11:2221-28, 2000). The skilled artisan will appreciate that any number of thermostable ligases, including DNA ligases and RNA ligases, can be obtained from thermophilic or hyperthermophilic organisms, for example, certain species of eubacteria and archaea, including viruses that infect such thermophilic or hyperthermophilic organisms; and that such ligases can be employed in the disclosed methods and kits.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucl. Acids Res., 27:875-81 (1999); Gryaznov and Letsinger, Nucl. Acids Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucl. Acids Res. 20:3005-09 (1992); Sievers and von Kiedrowski, Nature 369:221-24 (1994); Liu and Taylor, Nucl. Acids Res. 26:3300-04 (1999); Wang and Kool, Nucl. Acids Res. 22:2326-33 (1994); Purmal et al., Nucl. Acids Res. 20:3713-19 (1992); Ashley and Kushlan, Biochemistry 30:2927-33 (1991); Chu and Orgel, Nucl. Acids Res. 16:3671-91 (1988); Sokolova et al., FEBS Letters 232:153-55 (1988); Naylor and Gilham, Biochemistry 5:2722-28 (1966); James and Ellington, Chem. & Biol. 4:595-605 (1997); and U.S. Pat. No. 5,476,930.

Photoligation using light of an appropriate wavelength as a ligation agent is also within the scope of the current teachings. In certain embodiments, photoligation comprises probes comprising nucleotide analogs, including but not limited to, 4-thiothymidine (s⁴T), 5-vinyluracil and its derivatives, or combinations thereof. In certain embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In certain embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

When used in the context of the present teachings, “suitable for ligation” refers to a first cleavage probe and a corresponding fragment of the second cleavage probe or a first ligation probe and a corresponding second ligation probe, each comprising an appropriately reactive group, typically on their respective ends that face each other. Exemplary pairs of reactive groups include, but are not limited to: a nucleotide 3′-hydroxyl group on the 3′ end of the first probe and a nucleotide 5′-phosphate group on the 5′ end of the second probe or a hybridized fragment of a second probe; phosphorothioate and tosylate or iodide; esters and hydrazide; RC(O)S⁻, haloalkyl, or RCH₂S and α-haloacyl; thiophosphoryl and bromoacetoamido groups. Additionally, in certain embodiments, first probe and the corresponding second probe are hybridized to the corresponding target sequence or the corresponding ligation product such that the 3′ end of the first probe and the 5′ end of the second probe are immediately adjacent or can be rendered immediately adjacent by gap-filling.

The term “ligation product surrogate” or “surrogate” as used herein refers to any molecule or moiety whose detection or identification indicates the existence of a corresponding ligation product and thus, the presence of a particular target nucleotide. Exemplary ligation product surrogates include but are not limited to, digested ligation products; amplified ligation products; digested amplified ligation products; moieties cleaved or released from a ligation product or ligation product surrogate; complementary strands or counterparts of a ligation product or ligation product surrogate; reporter probes that are or were annealed to a ligation product or another ligation product surrogate, including but not limited to cleavage and amplification products thereof; hybridization tag complements that are or were annealed to a ligation product or another ligation product surrogate, including but not limited to ZipChute™ reagents (typically a molecule or complex comprising a hybridization tag complement, a mobility modifier, and a reporter group, generally a fluorescent reporter group; see, e.g., Applied Biosystems Part Number 4344467 Rev. C; see also U.S. Provisional Patent Application Ser. No. 60/517,470) or parts of hybridization tag complements; and the like.

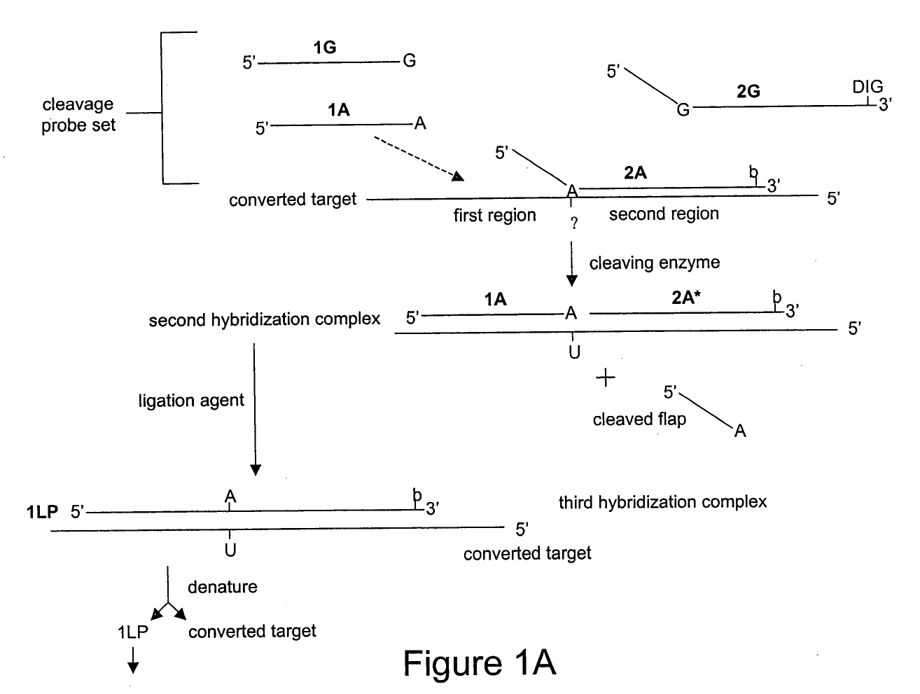

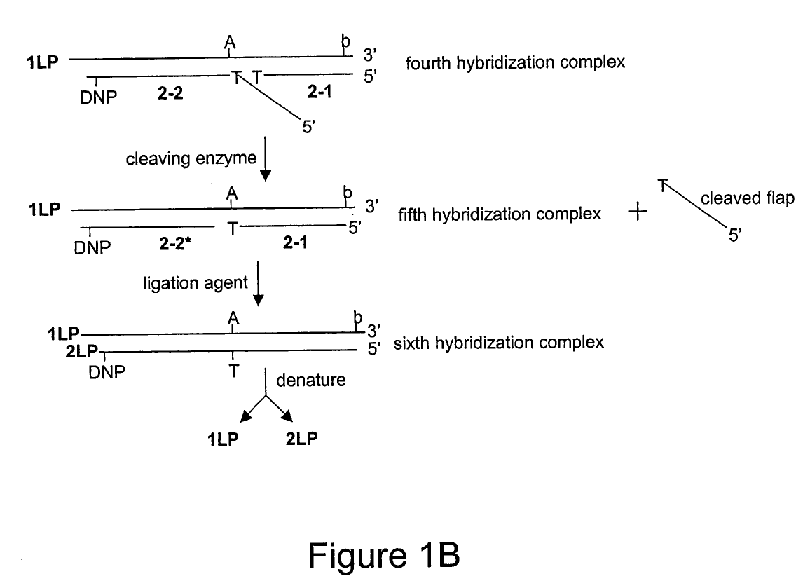

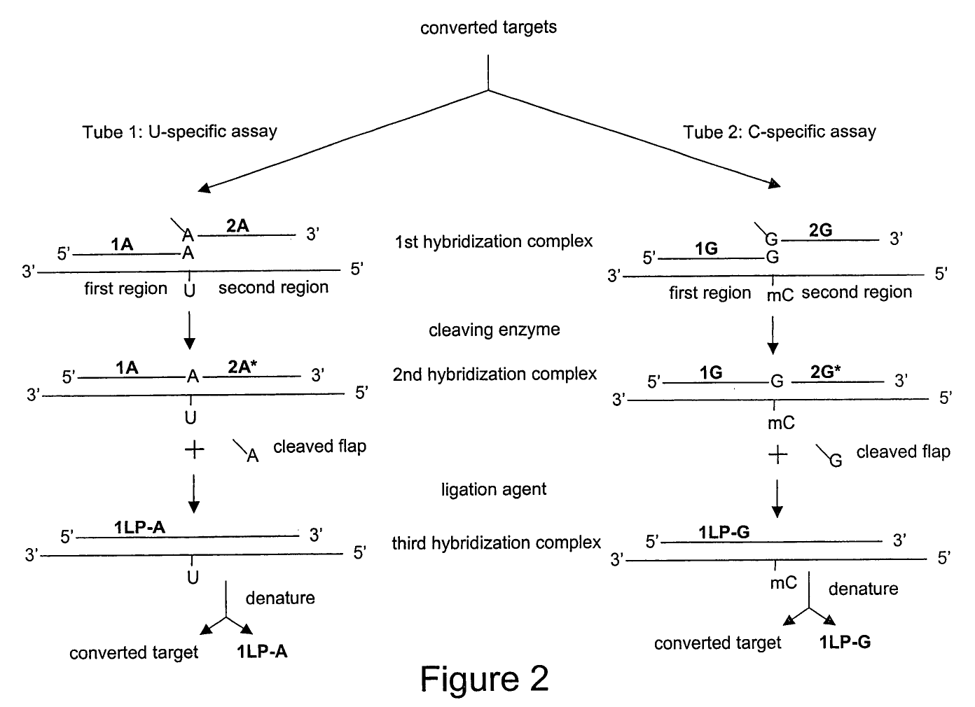

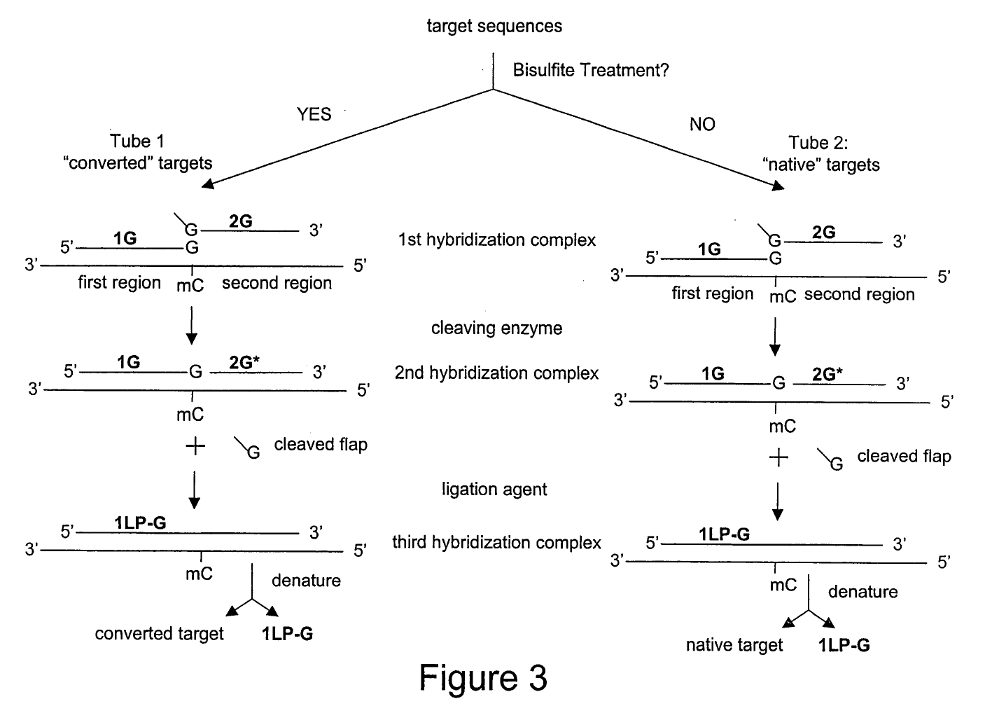

As used herein, “ligation product yield”, “ligation yield”, or “yield” are relative terms that can be used interchangeably and are determined by evaluating one or more measurable parameter of a ligation product or its surrogate. In certain embodiments, experimentally obtained ligation yields are used to create ligation yield ratios. The terms “ligation yield ratio”, “ligation ratio”, or “ratio” are also interchangeable terms and are obtained by comparing one or more quantifiable parameter of a first ligation product with the same quantifiable parameter(s) of a second ligation product generated under the same or similar conditions. For example but not limited to, comparing the ligation yield of one cleavage probe pair designed to interrogate an unconverted target nucleotide in a converted target sequence (such as the 1 G-2G probe pair of FIG. 1A) with the ligation yield of a second probe pair of the same cleavage probe set designed to interrogate the corresponding converted target nucleotide (i.e., a related probe pair, such as the 1A-2A probe pair of FIG. 1A) in the same coupled cleavage-ligation assay and creating a ligation ratio; comparing the ligation yield obtained with a probe pair in one assay with the ligation yield obtained with a related probe pair in a parallel assay using the same or similar sample (see, e.g., FIG. 2); or comparing the ligation ratio obtained using the same cleavage probe pair (such the 1 G-2G probe pair of FIG. 3) with (1) a converted sample and (2) the corresponding unconverted sample, in parallel or substantially parallel coupled cleavage-ligation assays (i.e., “treated” versus “untreated” for example, as shown in FIG. 3). Those in the art appreciate that numerous measurable parameters exist that can be used to compare the amounts of two or more ligation products generated under the same or similar conditions, for example but not limited to, ligation product peak height, integrated area under the ligation product curve, signal intensity, and threshold cycle (“C_{T}”, sometimes written as Cₜ), including without limitation, ΔC_{T} and ΔΔC_{T}. By evaluating the ligation yield or the ligation ratio, one can determine the degree of methylation of a target nucleotide. In certain embodiments, a ligation yield, a ligation ratio, or a ligation yield and a ligation ratio, are determined in “real-time”, i.e., as the reaction progresses and products accumulate (typically using methods analogous to quantitative PCR; see, e.g., Essentials of Real Time PCR, Applied Biosystems P/N 105622, 2002). In certain embodiments, a ligation yield, a ligation ratio, or a ligation yield and a ligation ratio are determined using end-point analyses, i.e., after the reaction has reached completion.

In certain embodiments, the ligation yield for a given ligation product or the ligation ratio for two ligation products is compared to at least one corresponding standard curve and the degree of target nucleotide methylation can be inferred. Standard curves for determining the degree of target nucleotide methylation can be generated, if desired, using pre-determined mixtures of methylated and non-methylated synthetic templates or gDNA as the target sequences in one or more of the disclosed coupled cleavage-ligation assays or ligation assays under standard conditions. Those in the art are familiar with generating and using standard curves and C_{T} values (see, e.g., Overholtzer et al., Proc. Natl. Sci. 100:11547-52, 2003; Osiowy, J. Clin. Micro. 40:2566-71, 2002; and ABI PRISM® 7700 Sequence Detection System User Bulletin #2, updated 10/2001, Applied Biosystems).

As used herein, the term “methylation state” refers to the presence or absence of a methyl group on a particular target nucleotide, generally but not always, a cytosine. The term “target nucleotide” refers to a specific nucleotide in a target sequence, the methylation state of which is sought to be determined, including without limitation, whether that nucleotide comprises a cytosine or a 5-methylcytosine; or an adenosine or a N⁶-methyladenosine. In certain embodiments, a target nucleotide is modified, for example but not limited to, bisulfite treatment of the corresponding target sequence. Thus, in certain embodiments comprising unconverted target sequences the target nucleotide is a cytosine or a 5-methylcytosine, while in other embodiments comprising converted target sequences, the target nucleotide can be 5-methylcytosine or uracil (i.e., a converted target nucleotide). In certain embodiments, a pivotal nucleotide is a target nucleotide. In certain embodiments, a pivotal nucleotide is a converted target nucleotide. The target nucleotide or the converted target nucleotide is typically interrogated by the first cleavage probe set and the complement of the target nucleotide or the complement of the converted target nucleotide is typically interrogated by the second cleavage probe set or the first ligation probe set.

A sample may contain a mixture of target sequences, some of which are methylated at a particular target nucleotide and some of which are not methylated at that target nucleotide. As used herein, the term “degree of methylation” refers to the relative number, percentage, or fraction of members of a particular target nucleotide species within a sample that are methylated compared to those members of that particular target nucleotide species that are not methylated.

The term “mobility-dependent analytical technique” as used herein, refers to any means for separating different molecular species based on differential rates of migration of those different molecular species in one or more separation techniques. Exemplary mobility-dependent analytical techniques include electrophoresis, chromatography, mass spectrometry, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682; PCT Publication No. WO 01/92579; D. R. Baker, Capillary Electrophoresis, Wiley-Interscience (1995); Biochromatography: Theory and Practice, M. A. Vijayalakshmi, ed., Taylor & Francis, London, U.K. (2003); Krylov and Dovichi, Anal. Chem. 72:111R-128R (2000); Swinney and Bornhop, Electrophoresis 21:1239-50 (2000); Crabtree et al., Electrophoresis 21:1329-35 (2000); and A. Pingoud et al., Biochemical Methods: A Concise Guide for Students and Researchers, Wiley-VCH Verlag GmbH, Weinheim, Germany (2002).

The term “mobility modifier” as used herein refers to a molecular entity, for example but not limited to, a polymer chain, that when added to an element (e.g., a probe, a primer, a ligation product, a ligation product surrogate, or combinations thereof) affects the mobility of the element to which it is hybridized or bound, covalently or non-covalently, in a mobility-dependent analytical technique.

As used herein, the term “Modification” refers to a substituted hydrocarbon, a ribonucleotide, an amide bond (including but not limited to a PNA or a pcPNA), a nucleotide analog, a groove binder, or combinations thereof. In certain embodiments, a probe comprises a Modification, sometimes referred to as a “Modified probe.” In certain embodiments, a Modification comprises a structure shown below, 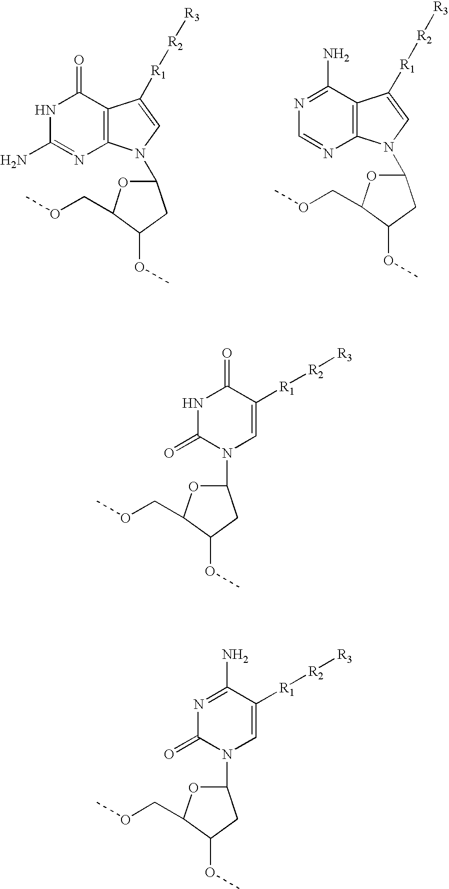
wherein: (a) R₁ comprises a hydrogen, alkyl, substituted alkyl, alkene, substituted alkene, alkyne, substituted alkyne, aromatic ring, substituted aromatic ring, heteroaromatic ring, substituted heteroaromatic ring, halogen, nitro, cyano, oxygen, substituted oxygen, nitrogen, substituted nitrogen, divalent sulfur, substituted divalent sulfur, sulfonate, sulfonate ester, aldehyde, ketone carbon with R₂, carboxylate carbon as carboxylic acid and ester with R₂, or combinations thereof; (b) R₂, a substituent on R₁, comprises at least one hydrogen, alkyl, substituted alkyl, alkene, substituted alkene, alkyne, substituted alkyne, aromatic ring, substituted aromatic ring, heteroaromatic ring, substituted heteroaromatic ring, halogen, nitro, cyano, alcohol, ether substituted with R₃, amine, secondary, tertiary, and quaternary amines substituted with R₃, amido substituted with R₃, thiol, thioether substituted with R₃, sulfonate, sulfonate ester substituted with R₃, phosphate and phosphate esters substituted with R₃, phosphonate and phosphonate esters substituted with R₃, aldehyde, ketone substituted with R₃, carboxylate, carboxylate esters substituted with R₃, carboxyamides substituted with R₃., or combinations thereof; and (c) R₃, a substituent on R₂, comprises a hydrogen, alkyl, substituted alkyl, alkene, substituted alkene, alkyne, substituted alkyne, aromatic ring, substituted aromatic ring, heteroaromatic ring, substituted heteroaromatic ring, halogen, nitro, cyano, alcohol, ether as defined in R₂, amine, secondary, tertiary, and quaternary amines as defined in R₂, amido as defined in R₂, thiol, thioether as defined in R₂, sulfonate, sulfonate ester as defined in R₂, phosphate and phosphate esters as defined in R₂, phosphonate and phosphonate esters as defined in R₂, aldehyde, ketone as defined in R₂, carboxylate, carboxylate esters as defined in R₂, carboxyamides as defined in R₂.

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick or Hoogsteen-type hydrogen bonds with a complementary nucleotide base. Exemplary nucleotide bases and analogs thereof include, but are not limited to, naturally-occurring nucleotide bases adenine, guanine, cytosine, 5 methyl-cytosine, uracil, thymine, and analogs of the naturally occurring nucleotide bases, including without limitation, 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2 ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT Published Application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different, —R, —OR, —NR₂ azide, cyanide or halogen groups, where each R is independently H, C₁-C₆ alkyl, C₂-C₇ acyl, or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT Published Application Nos. WO 98/22489, WO 98/39352, and WO 99/14226; and Braasch and Corey, Chem. Biol. 8:1-7, 2001). “LNA” or “locked nucleic acid” is a DNA analogue that is conformationally locked such that the ribose ring is constrained by a methylene linkage between the 2′-oxygen and the 3′- or 4′-carbon. The conformation restriction imposed by the linkage often increases binding affinity for complementary sequences and increases the thermal stability of such duplexes. Exemplary LNA sugar analogs within a polynucleotide include, but are not limited to, the structures: 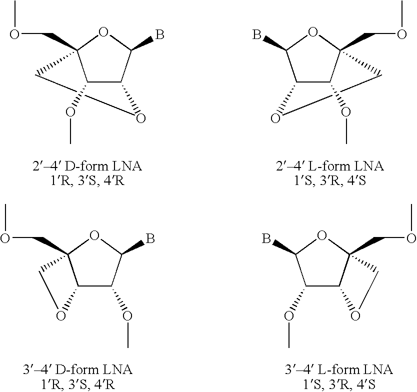
where B is any nucleobase.

The 2′- or 3′-position of ribose can be modified to include, without limitation, hydrogen, hydroxy, methoxy, ethoxy, allyloxy, isopropoxy, butoxy, isobutoxy, methoxyethyl, alkoxy, phenoxy, azido, cyano, amido, imido, amino, alkylamino, fluoro, chloro and bromo. Nucleotides include, but are not limited to, the natural D optical isomer, as well as the L optical isomer forms (see, e.g., Garbesi Nucl. Acids Res. 21:4159-65 (1993); Fujimori (1990) J. Amer. Chem. Soc. 112:7435; Urata, (1993) Nucleic Acids Symposium Ser. No. 29:69-70). When the nucleotide base is purine, e.g., A or G, the ribose sugar is attached to the N⁹-position of the nucleotide base. When the nucleotide base is pyrimidine, e.g. C, T, or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 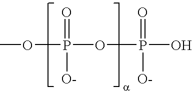
where α is an integer from 0 to 4. In certain embodiments, α is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. Reviews of nucleotide chemistry can be found in, among other places, Shabarova, Z. and Bogdanov, A. Advanced Organic Chemistry of Nucleic Acids, VCH, New York, 1994; and Blackburn and Gait.

The term “nucleotide analog”, as used herein, refers to embodiments in which the pentose sugar or the nucleotide base or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of nucleotide analog are monomers that can be polymerized into polynucleotide analogs in which the DNA/RNA phosphate ester or sugar phosphate ester backbone is replaced at least in part by a different type of inter-nucleotide linkage. Exemplary polynucleotide analogs include, but are not limited to, peptide nucleic acids (PNAs), in which the sugar phosphate backbone of the polynucleotide is replaced by a peptide backbone comprising at least one amide bond. It is to be understood that the term “PNA” as used herein, includes pseudocomplementary PNAs (pcPNAs) unless otherwise apparent from the context. (See, e.g., Datar and Kim, Concepts in Applied Molecular Biology, Eaton Publishing, Westborough, Mass., 2003, particularly at pages 74-75; Verma and Eckstein, Ann. Rev. Biochem. 67:99-134, 1998; Goodchild, Bioconj. Chem., 1:165-187, 1990; Braasch and Corey, Methods 23:97-107, 2001; Demidov et al., Proc. Natl. Acad. Sci. 99:5953-58, 1999).

As used herein, the terms “polynucleotide”, “oligonucleotide”, “nucleic acid”, and “nucleic acid sequence” are generally used interchangeably and include single-stranded and double-stranded polymers of nucleotide monomers, including 2′-deoxyribonucleotides (DNA) and ribonucleotides (RNA) linked by inter-nucleotide phosphodiester bond linkages, or inter-nucleotide analogs, and associated counter ions, e.g., H⁺, NH₄⁺, trialkylammonium, tetraalkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40, when they are sometimes referred to in the art as oligonucleotides, to several thousands of monomeric nucleotide units. Nucleic acid sequence are shown in the 5′ to 3′ orientation from left to right, unless otherwise apparent from the context or expressly indicated differently; and in such sequences, “A” denotes deoxyadenosine, “C” denotes deoxycytidine, “G” denotes deoxyguanosine, “T” denotes thymidine, and “U” denotes uridine, unless otherwise apparent from the context.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 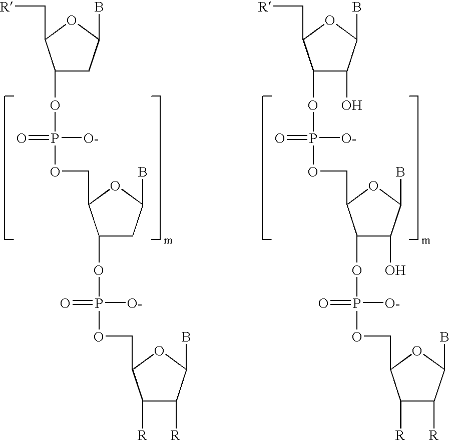
wherein each B is independently the base moiety of a nucleotide, e.g., a purine, a 7-deazapurine, a purine or purine analog substituted with one or more substituted hydrocarbons, a pyrimidine, a pyrimidine or pyrimidine analog substituted with one or more substituted hydrocarbons, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6) alkyl, (C2-C7) acyl or (C5-C14) aryl, cyanide, azide, or two adjacent R′ are taken together to form a bond such that the ribose sugar is 2′,3′-didehydroribose; and each R′ is independently hydroxyl or 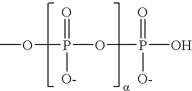
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “nucleic acid sequence”, “polynucleotide”, and “oligonucleotide” can also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog or at least one phosphate ester analog or at least one pentose sugar analog. Also included within the definition of nucleic acid analogs are nucleic acids in which the phosphate ester or sugar phosphate ester linkages are replaced with other types of linkages, such as N-(2-aminoethyl)-glycine amides and other amides (see, e.g., Nielsen et al., 1991, Science 254: 1497-1500; PCT Publication No. WO 92/20702; U.S. Pat. Nos. 5,719,262 and 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, J. Org. Chem. 52: 4202, 1987); methylene(methylimino) (see, e.g., Vasseur et al., J. Am. Chem. Soc. 114:4006, 1992); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., PCT Publication No. WO 92/20702; Nielsen, Science 254:1497-1500, 1991); and others (see, e.g., U.S. Pat. No. 5,817,781; Frier & Altman, Nucl. Acids Res. 25:4429, 1997 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. See also, Scheit, Nucleotide Analogs, John Wiley, New York, (1980); Englisch, Agnew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1999.

A “pivotal nucleotide” is the nucleotide being interrogated by a probe, for example but not limited to, a cleavage probe pair or a ligation probe. In certain embodiments, the pivotal nucleotide is a target nucleotide or a converted target nucleotide, for example but not limited to, when the nucleic acid being interrogated is a target sequence. In certain embodiments, the pivotal nucleotide is the nucleotide complement of the target nucleotide or the complement of the converted target nucleotide, for example but not limited to, when the nucleic acid being interrogated is a ligation product. The term “pivotal complement” refers to the nucleotide that is the hybridization partner or the potential hybridization partner of the pivotal nucleotide and is a component of the probe set being used to interrogate the pivotal nucleotide. For example but without limitation, as shown in FIG. 1A, when interrogating a cytosine target nucleotide of unknown methylation state in a converted target sequence, the pivotal complement of one cleavage probe pair is a guanine and the pivotal complement of the related cleavage probe pair of the illustrative probe set is an adenine (see, e.g., 1G+2G and 1A+2A, respectively in FIG. 1A). Typically, the pivotal complement is located at the 3′-end of the first probe, the 5′-end of the target region-binding portion of a second probe, or the 3′-end of a first probe and the 5′-end of the target region-binding portion of the corresponding second probe, but not always.

The term “reporter group” is used in a broad sense herein and refers to any identifiable tag, label, or moiety. The skilled artisan will appreciate that many different species of reporter groups can be used in the present teachings, either individually or in combination with one or more different reporter group. The term reporter group also encompasses an element of multi-element indirect reporter systems, including without limitation, affinity tags; and multi-element interacting reporter groups or reporter group pairs, such as fluorescent reporter group/quencher pairs, including without limitation, fluorescent quenchers and dark quenchers, also known as non-fluorescent quenchers (NFQ).

A “substituted hydrocarbon”, as that term is used herein, comprises a hydrocarbon where a hydrogen atom in the hydrocarbon assembly is replaced by: a hydrocarbon; a heterocyclic hydrocarbon; a substituted heterocyclic hydrocarbon; halogen; azide, cyanide, isocyanide, isocyanate, isothiocyanate, —OSO3—, —OSO3R, —SO3—, —SO3R, —OC(O)R, —OC(O)OR, —OR, —CO2R, —C(O)NR2, —NR2, —NRC(O)R, —N(C(O)R)2, —SR, —OP(O)(OR)2, —OP(O)(OR)R, —OP(O)R2, —P(O)(OR)2, —P(O)(OR)R, —P(O)R2, where R comprises hydrogen, hydrocarbon, heterocyclic hydrocarbon, substituted heterocyclic hydrocarbon, or substituted hydrocarbon. A hydrocarbon comprises an assembly of carbon atoms where any carbon valences not used for forming a bond with another carbon atom are used for bonding with hydrogen atoms. A hydrocarbon assembly comprises: a linear chain of carbon atoms where each of the carbon atoms is connected to a neighboring carbon atom by a single, double, or triple bond; a cyclic chain of carbon atoms where each of the carbon atoms is connected to at least two other carbon atom by a single, double, or in some unusual cases a triple bond; multiple cyclic chains of carbon atoms as described above where at least two of the cyclic chains share a common carbon-carbon single or multiple bond to form a fused ring system; multiple cyclic chains of carbon atoms as describe above where at least two cyclic chains are connected together by a carbon-carbon single or double bond, but where two bound cyclic chains do not share a common carbon-carbon single or double bond.

The term “target sequence” or “target” as used herein refers to a specific nucleic acid oligomer, typically genomic DNA (gDNA), that contains target nucleotides. A target nucleotide is that nucleotide in the target sequence that is interrogated by a probe set to determine its methylation state. Generally, a target nucleotide is a cytosine or a 5-methylcytosine in a CpG motif, but not always. For example but not limited to, certain embodiments, wherein the target nucleotide is an adenine or a N⁶-methyladenosine. While the target sequence is generally described as a single-stranded molecule, it is to be understood that double-stranded molecules that contain one or more target nucleotides are also considered target sequences. The term target sequence is generally used generically and can include non-converted target sequences or converted target sequences, unless expressly stated or otherwise apparent from the context. Target sequences can include both naturally occurring and synthetic sequences.

A wide variety of nucleic acid isolation techniques are well known in the art and are useful in obtaining target sequences for use in the teachings herein. Detailed descriptions of such techniques can be found in, among other places, Ausubel et al.; Rapley; and Sambrook et al.; see also, ABI PRISM™ 6100 Nucleic Acid PrepStation and ABI PRISM™ 6700 Automated Nucleic Acid Workstation, BloodPrep™ Chemistry kit, and NucPrep™ Chemistry kit, including the corresponding manuals and manufacturer's protocols (Applied Biosystems).

The term “threshold cycle” or “C_{T}” is used in reference to quantitative or real-time analysis methods and indicates the fractional cycle number at which the amount of product, such as the products of a coupled cleavage-ligation reaction or a ligation reaction including without limitation ligation products, cleaved flaps, or ligation product surrogates, reaches a fixed threshold or limit. Threshold cycles can be manually set by the user or determined by the software of a real-time instrument including without limitation, the ABI PRISM® 7700 Sequence Detection System, the ABI PRISM® 7900 HT Sequence Detection System, the ABI PRISM® 7300 Real-Time PCR System (Applied Biosystems), the Smart Cycler System (Cepheid, distributed by Fisher Scientific), the LightCycler™ System (Roche Molecular), or the Mx4000 (Stratagene, La Jolla, Calif.). Detailed descriptions of threshold cycles and their use, including without limitation ΔC_{T} and ΔΔC_{T}, can be found in, among other places, the ABI PRISM® 7700 Sequence Detection System User Bulletin #2, 2001. Those in the art will appreciate that while the threshold cycle concept generally refers to quantitative PCR technology, it can be readily adapted to the real-time quantitation of products generated during coupled cleavage-ligation reaction cycles or ligation cycles. In certain embodiments, such real-time quantitation comprises reporter probes; or intercalating dyes, including without limitation, ethidium bromide and SYBR Green I.

The terms “universal base” or “universal nucleotide” are generally used interchangeably herein and refer to a nucleotide analog (including nucleoside analogs) that can substitute for more than one of the natural nucleotides or natural bases in oligonucleotides. Universal bases typically contain an aromatic ring moiety that may or may not contain nitrogen atoms and generally use aromatic ring stacking to stabilize a duplex. In certain embodiments, a universal base may be covalently attached to the C-1′ carbon of a pentose sugar to make a universal nucleotide. In certain embodiments, a universal base does not hydrogen bond specifically with another nucleotide base. In certain embodiments, a nucleotide base may interact with adjacent nucleotide bases on the same nucleic acid strand by hydrophobic stacking. Universal nucleotides and universal bases include, but are not limited to, deoxy-7-azaindole triphosphate (d7AITP), deoxyisocarbostyril triphosphate (dICSTP), deoxypropynylisocarbostyril triphosphate (dPICSTP), deoxymethyl-7-azaindole triphosphate (dM7AITP), deoxylmPy triphosphate (dImPyTP), deoxyPP triphosphate (dPPTP), deoxypropynyl-7-azaindole triphosphate (dP7AITP), 3-methyl isocarbostyril (MICS), 5-methyl isocarbyl (5MICS), imidazole-4-carboxamide, 3-nitropyrrole, 5-nitroindole, hypoxanthine, inosine, deoxyinosine, 5-fluorodeoxyuridine, 4-nitrobenzimidizole, and PNA-bases, including pcPNA bases. Detailed descriptions of universal bases can be found in, among other places, Loakes, Nucl. Acids Res. 29:2437-47, 2001; Berger et al., Nucl. Acids Res. 28:2911-14, 2000; Loakes et al., J. Mol. Biol. 270:426-35, 1997; Verma and Eckstein, Ann. Rev. Biochem. 67:99-134, 1998; Published PCT Application No. US02/33619, and U.S. Pat. Nos. 6,433,134 and 6,433,134.

II. Certain Exemplary Components

The term “probe” as used herein, refers to an oligonucleotide comprising a sequence that is capable, under appropriate conditions, of selectively hybridizing with a region of a corresponding target sequence, a corresponding converted target sequence, a corresponding ligation product, or combinations thereof. The complementary sequences of the probes can be of any length suitable for use in the current teachings. Generally, the lengths should be sufficiently long to ensure specific hybridization to corresponding target regions, corresponding ligation product regions, or corresponding ligation product surrogate regions, but typically without significant cross-hybridization to non-corresponding/irrelevant nucleic acids. The terms probe and probes generally refer to cleavage probes and ligation probes. A probe may include Watson-Crick bases or modified bases, including but not limited to, a universal base, a Modification, and the AEGIS bases (from Eragen Biosciences), described, e.g., in U.S. Pat. Nos. 5,432,272; 5,965,364; and 6,001,983. Additionally, bases may be joined by a natural phosphodiester bond or a different chemical linkage. Different chemical linkages include, without limitation, amide linkages and LNA linkages, for example but not limited to, those described in published PCT Application Nos. WO 00/56748 and WO 00/66604. Probes can be prepared by any suitable means, including without limitation, automated synthesizers with standard resins or controlled pore glass (CPG). (See, e.g., ABI 3948 Nucleic Acid Synthesis and Purification System User's Manual (2002), Expedite 8900 Nucleic Acid Synthesis System User's Guide (2001), and PNA Chemistry for the Expedite 8900 Nucleic Acid Synthesis System User's Guide (2001), all from Applied Biosystems; Glen Research 2002 Catalog, User Guide to Modification and Labeling, 1999, and Glen Reports 16(2), 2003, all from Glen Research, Sterling, Va.; Braasch and Corey, Methods 23:97-107, 2001; and Blackburn and Gait).

In certain embodiments, a probe comprises a universal base. In certain embodiments, a probe comprises a multiplicity of universal bases. In other embodiments, the multiplicity of universal bases comprises at least two different universal bases in the probe. In certain embodiments, a first probe and a corresponding second probe comprise a universal base. In certain embodiments, a series of different probes that are designed for interrogating the same target nucleotide include degenerate bases relative to each other. In certain embodiments, a probe comprises a PNA, an LNA, a groove binder, or combinations thereof. Those in the art will understand that probes comprising PNAs, LNA, groove binders, or combinations, typically have higher Tm values than the corresponding probe lacking PNA, LNA, or groove binders,

Probes usually are part of a cleavage probe set or a ligation probe set, which typically include a first probe and a second probe. In certain embodiments, a probe set comprises at least two probe pairs, wherein each probe pair comprises a first probe and a corresponding second probe and wherein each probe pair in a probe set is designed to interrogate one of the possible nucleotides at a polymorphic pivotal nucleotide, for example but not limited to a C or a U target nucleotide in a converted target sequence or a G or an A pivotal nucleotide in the corresponding first ligation product (i.e., the complement of the target nucleotide present in the ligation product). The alternate first probes in a probe set comprising two or more probe pairs typically hybridize with the same region (or at least part of the same region) of the target sequence or ligation product, but differ in their respective pivotal complements and therefore their ability to specifically hybridize with the pivotal nucleotide. In certain embodiments, a first cleavage probe set is combined with an unconverted target sequence and a cleaving enzyme. In other embodiments, a first cleavage probe set is combined with a converted target sequence and a cleaving enzyme. Certain cleavage probe sets include one probe pair, while other cleavage probe sets comprise two or more probe pairs, depending at least in part, on the assay format. In certain embodiments, a second cleavage probe set is combined with a first ligation product and a cleaving enzyme. In certain embodiments, a target sequence is combined with a first cleavage probe set, a cleaving enzyme and a ligation agent in a single reaction vessel. In certain embodiments, the single reaction vessel further comprises a second cleavage probe set, a first ligation probe set, a second ligation probe set, a primer set, a polymerase, or combinations thereof. In certain embodiments, a first ligation product is combined with a first ligation probe set and a ligation agent. In certain embodiments, a second ligation product is combined with a second ligation probe set and a ligation agent. In certain embodiments, a reaction composition further comprises a polymerase and a hybridization complex and a gap-filling reaction is performed. Those in the art will appreciate that the order of adding various components to the reaction compositions of the disclosed methods is generally not limiting, unless otherwise apparent from the context.

A “first cleavage probe set” includes a probe pair comprising (1) a first probe that includes a portion that is complementary to a first target region and (2) a second probe comprising (a) a portion that is complementary to a second target region and (b) a flap portion (which may or may not be present initially). In certain embodiments, a first cleavage probe set comprises at least two probe pairs wherein each probe pair comprises a first cleavage probe and a corresponding second cleavage probe, for example but not limited to, one probe pair to interrogate a converted target nucleotide (i.e., “U”) in a converted target sequence and another probe pair to interrogate the unconverted target nucleotide (i.e., “C”) in the converted target sequence (see, e.g., the “A” probe pair, 1A and 2A, and the “G” probe pair, 1G and 2G, in FIG. 1A). The cleavage probes of the first cleavage probe set are designed to anneal to the first and second target regions of the target sequences or the converted target sequences, where the first and second target regions overlap by a nucleotide. However, target region overlaps of more than one nucleotide, for example but not limited to overlaps of 2, 3, 4, 5, 6, 8, 10, 12, and 15 nucleotides are also contemplated. Typically, the segment of the target sequence or the converted target sequence where the first and second target regions overlap comprises the target nucleotide or the converted target nucleotide, as appropriate. Thus, in certain embodiments, the first and second cleavage probes of a first cleavage probe set hybridize with the target sequence to form a “first hybridization complex”, wherein the 3′-end of the hybridized first probe will overlap with the 5′-end of the second target region-binding portion of the hybridized second probe, as shown in FIG. 1A.

The first hybridization complex, comprising the target, the first cleavage probe, and the corresponding second cleavage probe including the flap portion, typically serves as a suitable reaction substrate for a cleaving enzyme, and when the flap portion of the second cleavage probe is cleaved, a “second hybridization complex” is formed, comprising the first ligation probe, a fragment of the second ligation probe, and the target sequence, as shown in FIG. 1A. Flap cleavage typically generates a hybridized fragment of the second cleavage probe comprising a 5′ phosphate group that is hybridized on the target sequence adjacent to the corresponding first cleavage probe (shown as 2* in FIG. 1A). Thus, the first cleavage probe and the adjacently hybridized second cleavage probe fragment are suitable for ligation when the ligation agent comprises a ligase, provided that the 3′-end of the upstream probe comprises a nucleotide 3′ hydroxyl group. A ligation product is generated when such a first probe and second probe fragment are combined with a ligase under appropriate conditions and a “third hybridization complex” is formed, comprising the first ligation product and the target sequence, as shown in FIG. 1A. In certain embodiments, the second cleavage probe does not initially comprise a flap portion. Thus when a probe pair comprising such a second cleavage probe are reacted with the corresponding target sequence, the 3′-end of the first cleavage probe and the 5′-end of the second cleavage probe are adjacently hybridized, similar to a second hybridization complex (but without first forming the first hybridization complex). Subsequently, the 3′-end of the hybridized first cleavage probe can be extended by a polymerase and displace the 5′-end of the hybridized second cleavage probe, wherein the displaced 5′-end of the second cleavage probe becomes the flap portion of the second cleavage probe and a first hybridization complex is formed and can serve as a reaction substrate for a cleaving enzyme. Typically, a first probe extension step is performed in the presence of 1, 2, or 3 nucleotide triphosphate species, but not all four.

A “second cleavage probe set” is similar to the first cleavage probe set except that the probes of the second cleavage probe set are designed to specifically hybridize with regions of the first ligation product, not the target sequence, and the overlap region typically comprises the complement of the target nucleotide or the complement of the converted target nucleotide in the first ligation product. Similar to the first cleavage probe set, the overlap is at least one nucleotide and can be 2, 3, 4, 5, 6, 8, 10, 12, or 15 nucleotides. Extending the first cleavage probe of the second cleavage probe set is also contemplated. In certain embodiments, a second cleavage probe set comprises at least two probe pairs, wherein each probe pair comprises a first cleavage probe and a corresponding second cleavage probe, for example but not limited to, one probe pair to interrogate the complement of the converted target nucleotide (i.e., “A”) in a first ligation product and another probe pair to interrogate the complement of the corresponding unconverted target nucleotide (i.e., “G”) in the alternate first ligation product. In certain embodiments, a second cleavage probe set is combined with the first ligation product to form a “fourth hybridization complex”, that serves as a reaction substrate in a cleavage reaction to generate a cleaved flap and form a “fifth hybridization complex”, comprising the first cleavage probe of the second cleavage probe set (shown as 2-1 in FIG. 1B), a fragment of the second cleavage probe (shown as 2-2* in FIG. 1B), and the first ligation product. In the presence of a suitable ligation agent and under appropriate conditions, a “sixth hybridization complex” is formed comprising the first ligation product and a second ligation product (see, e.g., FIG. 1B). When the third and sixth hybridization complexes are denatured, the target sequence and first ligation product, or the first and second ligation products, respectively, are released and can be subjected to additional cycles of coupled cleavage-ligation reactions or ligation reactions, including without limitation, cycling between different reaction temperatures, such as with a thermocycler. Alternatively, the released first or second ligation products can be detected and the degree of target nucleotide methylation determined. In certain embodiments, the third hybridization complex, the sixth hybridization complex, or the third and the sixth hybridization complex are detected and the degree of target nucleotide methylation determined.

The ligation probe sets, in contrast, typically comprise a first ligation probe and a second ligation probe that are designed to specifically hybridize with regions of one or more ligation products. In certain embodiments, a ligation probe set comprises at least two probe pairs, wherein each probe pair comprises a first ligation probe and a corresponding second ligation probe, for example but not limited to, a first ligation probe set with one probe pair to interrogate the complement of the converted target nucleotide (i.e., “A”) in a first ligation product and another probe pair to interrogate the complement of the corresponding unconverted target nucleotide (i.e., “G”) in the alternate first ligation product. In certain embodiments, a first ligation probe set is combined with the first ligation product to form a “seventh hybridization complex”, comprising a first ligation probe and a second ligation probe annealed to the first ligation product. When combined with a suitable ligation agent under appropriate conditions, a third ligation product is generated, and an “eighth hybridization complex” is formed comprising a first ligation product annealed to the third ligation product. In certain embodiments, the third ligation product is combined with a second ligation probe set to form a “ninth hybridization complex” and, in the presence of a suitable ligation agent and under appropriate conditions, a fourth ligation product is generated and a “tenth hybridization complex” is formed, comprising the third ligation product annealed to the fourth ligation product.

When the eighth and tenth hybridization complexes are denatured, the first and third ligation products, or the second and fourth ligation products, respectively, are released and can be subjected to additional cycles of cleavage or ligation reactions. Alternatively, the released ligation products can be detected and the degree of target nucleotide methylation determined. In certain embodiments, the eighth hybridization complex, the tenth hybridization complex, or the eighth and the tenth hybridization complex are detected and the degree of target nucleotide methylation determined.

In certain embodiments, a first ligation product and the corresponding second ligation product comprise the same hybridization tag or the same affinity tag. In certain embodiments, a first ligation product comprising a hybridization tag and the corresponding second ligation product comprising the same hybridization tag are bound to a particular Substrate address comprising the corresponding hybridization tag complement and the first ligation product and the corresponding second ligation product are detected together at that Substrate address.

In certain embodiments, a first ligation product and the corresponding second ligation product comprise the same reporter probe-binding portion, such that under appropriate conditions, both the first ligation product and the corresponding second ligation product can be detected when combined with the corresponding reporter probe, for example but not limited to a real-time detection method.

Certain of the disclosed methods comprise a multiplicity of different probe sets for determining the degree of methylation of a multiplicity of different target nucleotides, including converted target nucleotides. Certain embodiments comprise a multiplex cleavage reaction or a multiplex ligation reaction. In certain embodiments, a multiplex cleavage reaction and a multiplex ligation reaction are performed in the same vessel, including without limitation, a tube; a multi-well plate, such as a 96-well, a 384-well, a 1536-well plate; and a microfluidic device, for example but not limited to, the TaqMan® Low Density Array (Applied Biosystems). In certain embodiments, a multiplex cleavage reaction, a multiplex ligation reaction, or a multiplex cleavage-ligation reaction are performed in the same reaction vessel comprising converted target sequences or unconverted target sequences. In certain embodiments, two or more multiplex coupled cleavage-ligation reactions comprising converted target sequences are performed in parallel or substantially in parallel (see, e.g., FIG. 2) in different wells of the same multi-well plate, different chambers of the same microfluidic device, and so forth. In certain embodiments, a multiplex cleavage-ligation reaction comprising unconverted target sequences is performed in parallel or essentially in parallel with a multiplex cleavage-ligation reaction comprising converted target sequences, wherein at least one cleavage probe set is common to both cleavage-ligation reactions (see, e.g., FIG. 3). In certain embodiments, a multiplicity of different target nucleotides are interrogated in the same reaction (i.e., not in a multiplicity of parallel reactions) using a multiplicity of probe pairs, wherein a probe pair corresponds to each target nucleotide being interrogated and wherein the ligation product of each probe pair is uniquely identifiable. In certain embodiments, a cleaving reaction, a ligation reaction, an amplification reaction, or combinations thereof, are automated or semi-automated, using an instrument or robotics.

The sequence-specific portions of cleavage probes are of sufficient length to permit specific annealing to complementary regions of corresponding target sequences, corresponding converted target sequences, or ligation products, as appropriate. The sequence-specific portions of ligation probes are of sufficient length to permit specific annealing to complementary regions of corresponding ligation products. In certain embodiments, the complementary portion of a probe contains a mismatch relative to the region of the target or ligation product to which it hybridizes, but optimally does not cross-react with other sequences. Likewise, primers are of sufficient length to permit specific hybridization to complementary sequences in corresponding ligation products, corresponding ligation product surrogates, or combinations thereof. The criteria for designing sequence-specific nucleic acid probes (including without limitation, cleavage probes, ligation probes, and reporter probes) and primers are well known to those in the art. In certain embodiments, a probe or a primer comprises at least one region that is complementary with the corresponding sequences in a target sequence, a converted target sequence, a ligation product, a ligation product surrogate, or combinations thereof. Detailed descriptions of nucleic acid probe and primer design can be found in, among other places, Diffenbach and Dveksler, PCR Primer, A Laboratory Manual, Cold Spring Harbor Press (1995); R. Rapley, The Nucleic Acid Protocols Handbook (2000), Humana Press, Totowa, N.J. (“Rapley”); Schena; and Kwok et al., Nucl. Acid Res. 18:999-1005 (1990). Primer and probe design software programs are also commercially available, including without limitation, Primer Express, Applied Biosystems; Primer Premier and Beacon Designer software, PREMIER Biosoft International, Palo Alto, Calif.; Primer Designer 4, Sci-Ed Software, Durham, N.C.; Primer Detective, ClonTech, Palo Alto, Calif.; Lasergene, DNASTAR, Inc., Madison, Wis.; Oligo software, National Biosciences, Inc., Plymouth, Minn.; iOligo, Caesar Software, Portsmouth, N.H.; and RTPrimerDB on the world wide web at realtimeprimerdatabase.ht.st or at medgen31.urgent.be/primerdatabase/index (see also, Pattyn et al., Nucl. Acid Res. 31:122-23, 2003).

Those in the art understand that the cleavage and ligation probes and the cleavage and ligation probe sets that are suitable for use with the disclosed methods and kits can be identified empirically using the current teachings and routine methods known in the art, without undue experimentation. For example, suitable probes and probe sets can be obtained by selecting appropriate target nucleotides and target nucleotide sequences by searching relevant scientific literature, including but not limited to appropriate databases (see, e.g., DNA Methylation Database (MethDB), on the web at methdb.de or methdb.net; CpG Island Searcher, on the web at cpgislands.com; the NCBI Entrez Nucleotide database), or by experimental analysis. When target sequences of interest are identified, test probes can be synthesized using well known oligonucleotide synthesis and organic chemistry techniques (see, e.g., Current Protocols in Nucleic Acid Chemistry, Beaucage et al., eds., John Wiley & Sons, New York, N.Y., including updates through June 2004 (“Beaucage et al.”); Blackburn and Gait; Glen Research 2002 Catalog, Sterling, Va.; and Synthetic Medicinal Chemistry 2003/2004, Berry and Associates, Dexter, Mich.). Test cleavage probes or cleavage probe sets are employed in the disclosed assays using appropriate target sequences or appropriate ligation products and their suitability for interrogating the corresponding pivotal nucleotide can be evaluated. Test ligation probes or ligation probe sets are employed in the disclosed assays using appropriate first ligation products or second ligation products and their suitability for interrogating the corresponding ligation product is evaluated. Those in the art will appreciate that the melting temperature (Tm) of a nucleotide probe can be increased by, among other things, incorporating a minor groove binder, substituting a corresponding PNA or LNA for a nucleotide (i.e., a chimeric probe), or using a PNA oligomer probe or LNA oligomer probe, with or without a groove binder.

In certain embodiments, a cleavage probe or a ligation probe comprises an additional component, including but not limited to, a primer-binding portion, a reporter probe-binding portion, a reporter group, a hybridization tag, a mobility modifier, an affinity tag, or combinations thereof. In certain embodiments, such additional components are within the sequence-specific portion, coextensive with the sequence-specific portion, overlaps at least part of the sequence-specific portion, or combinations thereof. In certain embodiments, a cleavage probe, a ligation probe, a ligation product, a cleaved flap, or combinations thereof, comprise a reporter group. In certain embodiments, a reporter group emits a fluorescent, a chemiluminescent, a bioluminescent, a phosphorescent, or an electrochemiluminescent signal. Exemplary reporter groups include, but are not limited to fluorophores, radioisotopes, chromogens, enzymes, antigens including but not limited to epitope tags, semiconductor nanocrystals such as quantum dots, heavy metals, dyes, phosphorescence groups, chemiluminescent groups, electrochemical detection moieties, affinity tags, binding proteins, phosphors, rare earth chelates, transition metal chelates, near-infrared dyes, including but not limited to, “Cy.7.5Ph.NCS,” “Cy.7.OphEt.NCS,” “Cy7.OphEt.CO₂Su”, and IRD800 (see, e.g., J. Flanagan et al., Bioconjug. Chem. 8:751-56 (1997); and DNA Synthesis with IRD800 Phosphoramidite, LI-COR Bulletin #111, LI-COR, Inc., Lincoln, Nebr.), electrochemiluminescence labels, including but not limited to, tris(bipyridal) ruthenium (II), also known as Ru(bpy)₃²⁺, Os(1,10-phenanthroline)₂bis(diphenylphosphino)ethane²⁺, also known as Os(phen)₂(dppene)²⁺, luminol/hydrogen peroxide, Al(hydroxyquinoline-5-sulfonic acid), 9,10-diphenylanthracene-2-sulfonate, and tris(4-vinyl-4′-methyl-2,2′-bipyridal) ruthenium (II), also known as Ru(v-bpy₃²⁺), and the like.

The term reporter group also encompasses an element of multi-element indirect reporter systems, including without limitation, affinity tags such as biotin:avidin, antibody:antigen, ligand:receptor including but not limited to binding proteins and their ligands, and the like, in which one element interacts with one or more other elements of the system in order to effect the potential for a detectable signal. Exemplary multi-element reporter systems include an oligonucleotide comprising a biotin reporter group and a streptavidin-conjugated fluorophore, or vice versa; an oligonucleotide comprising a DNP reporter group and a fluorophore-labeled anti-DNP antibody; and the like. In certain embodiments, reporter groups, particularly multi-element reporter groups, are not necessarily used for detection, but serve as affinity tags for isolation/separation, for example but not limited to, a biotin reporter group and a streptavidin-coated Substrate, or vice versa; a digoxygenin reporter group and a Substrate comprising an anti-digoxygenin antibody or a digoxygenin-binding aptamer; a DNP reporter group and a Substrate comprising an anti-DNP antibody or a DNP-binding aptamer; and the like. Detailed protocols for attaching reporter groups to oligonucleotides, polynucleotides, peptides, antibodies and other proteins, mono-, di- and oligosaccharides, organic molecules, and the like can be found in, among other places, G. T. Hermanson, Bioconjugate Techniques, Academic Press, San Diego, 1996; Beaucage et al.; Molecular Probes Handbook; and Pierce Applications Handbook and Catalog 2003-2004, Pierce Biotechnology, Rockford, Ill., 2003 (“Pierce Applications Handbook”).

Multi-element interacting reporter groups are also within the scope of the term reporter group, such as fluorophore-quencher pairs, including without limitation fluorescent quenchers and dark quenchers (also known as non-fluorescent quenchers). A fluorescent quencher can absorb the fluorescent signal emitted from a fluorophore and after absorbing enough fluorescent energy, the fluorescent quencher can emit fluorescence at a characteristic wavelength, e.g., fluorescent resonance energy transfer. For example without limitation, the FAM-TAMRA pair can be illuminated at 492 nm, the excitation peak for FAM, and emit fluorescence at 580 nm, the emission peak for TAMRA. A dark quencher, appropriately paired with a fluorescent reporter group, absorbs the fluorescent energy from the fluorophore, but does not itself fluoresce. Rather, the dark quencher dissipates the absorbed energy, typically as heat. Exemplary dark or nonfluorescent quenchers include Dabcyl, Black Hole Quenchers, Iowa Black, QSY-7, AbsoluteQuencher, Eclipse non-fluorescent quencher, metal clusters such as gold nanoparticles, and the like. Certain dual-labeled probes comprising fluorophore-quencher pairs can emit fluorescence when the members of the pair are physically separated, for example but without limitation, nuclease probes such as TaqMan® probes. Other dual-labeled probes comprising fluorophore-quencher pairs can emit fluorescence when the members of the pair are spatially separated, for example but not limited to hybridization probes such as molecular beacons or extension probes such as Scorpion primers. Fluorophore-quencher pairs are well known in the art and used extensively for a variety of reporter probes (see, e.g., Yeung et al., BioTechniques 36:266-75, 2004; Dubertret et al., Nat. Biotech. 19:365-70, 2001; and Tyagi et al., Nat. Biotech. 18:1191-96, 2000).

In certain embodiments, a reporter group comprises an electrochemiluminescent moiety that can, under appropriate conditions, emit detectable electrogenerated chemiluminescence (ECL). In ECL, excitation of the electrochemiluminescent moiety is electrochemically driven and the chemiluminescent emission can be optically detected. Exemplary electrochemiluminescent reporter group species include: Ru(bpy)₃²⁺ and Ru(v-bpy)₃²⁺ with emission wavelengths of 620 nm; Os(phen)₂(dppene)²⁺ with an emission wavelength of 584 nm; luminol/hydrogen peroxide with an emission wavelength of 425 nm; Al(hydroxyquinoline-5-sulfonic acid) with an emission wavelength of 499 nm; and 9,10-diphenylanothracene-2-sulfonate with an emission wavelength of 428 nm; and the like. Forms of these three electrochemiluminescent reporter group species that are modified to be amenable to incorporation into probes are commercially available or can be synthesized without undue experimentation using techniques known in the art. For example, a Ru(bpy)₃²⁺ N-hydroxy succinimide ester for coupling to nucleic acid sequences through an amino linker group has been described (see, U.S. Pat. No. 6,048,687); and succinimide esters of Os(phen)₂(dppene)²⁺ and Al(HQS)₃³⁺ can be synthesized and attached to nucleic acid sequences using similar methods. The Ru(bpy)₃²⁺ electrochemiluminescent reporter group can be synthetically incorporated into nucleic acid sequences using commercially available ru-phosphoramidite (IGEN International, Inc., Gaithersburg, Md.) (see, e.g., Osiowy, J. Clin. Micro. 40:2566-71, 2002).

Additionally other polyaromatic compounds and chelates of ruthenium, osmium, platinum, palladium, and other transition metals have shown electrochemiluminescent properties. Detailed descriptions of ECL and electrochemiluminescent moieties can be found in, among other places, A. Bard and L. Faulkner, Electrochemical Methods, John Wiley & Sons (2001); M. Collinson and M. Wightman, Anal. Chem. 65:2576 (1993); D. Brunce and M. Richter, Anal. Chem. 74:3157 (2002); A. Knight, Trends in Anal. Chem. 18:47 (1999); B. Muegge et al., Anal. Chem. 75:1102 (2003); H. Abrunda et al., J. Amer. Chem. Soc. 104:2641 (1982); K. Maness et al., J. Amer. Chem. Soc. 118:10609 (1996); M. Collinson and R. Wightman, Science 268:1883 et seq. (1995); and U.S. Pat. No. 6,479,233 (see also, O'Sullivan et al., Nucl. Acids Res. 30:el 14, 2002 for a discussion of phosphorescent lanthanide and transition metal reporter groups).

The term “Substrate” as used herein refers to one or more surfaces that a ligation product, a cleaved flap, a hybridization tag complement, or combinations thereof, can interact with or bind to, either directly or indirectly. A “reaction substrate,” by contrast, is a component of an enzyme-mediated reaction, such as an enzyme-substrate interaction. Substrate surfaces can be planar, spherical, circular, or any of a variety of topographies, including combinations of topographies on the same surface. In certain embodiments, various types of particles, beads, and microspheres can serve as suitable Substrates, including without limitation, magnetic beads, paramagnetic beads, coated beads, metallic particles, latex beads, acylamide beads, polyacrylamide beads, and reflective metallic microcylinders. Those in the art will appreciate that the suitability of a particular Substrate, including its topography and composition, typically depends on the separation or detection technique(s) employed. In certain embodiments, Substrates comprise oligonucleotides, including without limitation hybridization tag complements; PNA oligomers; LNA oligomers; chimeric molecules comprising at least two of the following: a deoxyribonucleotide, a ribonucleotide, a PNA monomer, an LNA monomer, and a nucleotide analog; antibodies; aptamers; affinity tags; or combinations thereof.

In certain embodiments, a cleavage probe, a ligation probe, a ligation product, or combinations thereof, comprise a mobility modifier. Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543; Grossman et al., Nucl. Acids Res. 22:4527-34, 1994). In certain embodiments, a multiplicity of probes exclusive of mobility modifiers, a multiplicity of primers exclusive of mobility modifiers, a multiplicity of ligation products exclusive of mobility modifiers, a multiplicity of ligation product surrogates exclusive of mobility modifiers, or combinations thereof, have the same or substantially the same mobility in a mobility-dependent analytical technique.

In certain embodiments, a multiplicity of probes, a multiplicity of primers, a multiplicity of ligation products, a multiplicity of ligation product surrogates, or combinations thereof, have substantially similar distinctive mobilities, for example but not limited to, when a multiplicity of elements comprising mobility modifiers have substantially similar distinctive mobilities so they can be bulk separated or they can be separated from other elements comprising mobility modifiers with different distinctive mobilities. In certain embodiments, a multiplicity of probes comprising mobility modifiers, a multiplicity of primers comprising mobility modifiers, a multiplicity of ligation products comprising mobility modifiers, a multiplicity of ligation product surrogates comprising mobility modifiers, or combinations thereof, have different distinctive mobilities.

In certain embodiments, a mobility modifier comprises a nucleotide polymer chain, including without limitation, an oligonucleotide polymer chain or a polynucleotide polymer chain. For example but not limited to a series of additional non-target sequence-specific nucleotides in a probe such as “TTTT” or nucleotide spacers (see e.g., Tong et al., Nat. Biotech. 19:756-759 (2001)). In certain embodiments, a mobility modifier comprises a non-nucleotide polymer chain. Exemplary non-nucleotide polymer chains include, without limitation, peptides, polypeptides, polyethylene oxide (PEO), or the like. In certain embodiments, a polymer chain comprises a substantially uncharged, water-soluble chain, such as a chain composed of a PEO unit; a polypeptide chain; or combinations thereof.

The polymer chain can comprise a homopolymer, a random copolymer, a block copolymer, or combinations thereof. Furthermore, the polymer chain can have a linear architecture, a comb architecture, a branched architecture, a dendritic architecture (e.g., polymers containing polyamidoamine branched polymers, Polysciences, Inc. Warrington, Pa.), or combinations thereof. In certain embodiments, a polymer chain is hydrophilic, or at least sufficiently hydrophilic when hybridized or bound to an element to ensure that the element-mobility modifier is readily soluble in aqueous medium. Where the mobility-dependent analytical technique is electrophoresis, in certain embodiments, the polymer chains are uncharged or have a charge/subunit density that is substantially less than that of its corresponding element.

The synthesis of polymer chains useful as mobility modifiers will depend, at least in part, on the nature of the polymer. Methods for preparing suitable polymers generally follow well-known polymer subunit synthesis methods. These methods, which involve coupling of defined-size, multi-subunit polymer units to one another, either directly or through charged or uncharged linking groups, are generally applicable to a wide variety of polymers, such as PEO, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of PEO units alternating with polypropylene units. Polypeptides of selected lengths and amino acid composition, either homopolymer or mixed polymer, can be synthesized by standard solid-phase methods (see, e.g., Int. J. Peptide Protein Res., 35: 161-214, 1990).

One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (see, e.g., U.S. Pat. No. 4,914,210; see also, U.S. Pat. No. 5,777,096).

The term “reporter probe” refers to a biomolecule, typically an oligonucleotide, that binds to or anneals with a ligation product, a ligation product surrogate, a cleaved flap, or combinations thereof, and can be used to determine the degree of methylation of a target nucleotide. Most reporter probes can be categorized based on their mode of action, for example but not limited to: nuclease probes, including without limitation TaqMan® probes and the like (see, e.g., Livak, Genetic Analysis: Biomolecular Engineering 14:143-149, 1999; Yeung et al., BioTechniques 36:266-75, 2004); extension probes such as scorpion primers, Lux™ primers, Amplifluors, and the like; hybridization probes such as molecular beacons, Eclipse probes, light-up probes, pairs of singly-labeled reporter probes, hybridization probe pairs, and the like; or combinations thereof. In certain embodiments, reporter probes comprise an amide bond, an LNA, a universal base, or combinations thereof, and include stem-loop and stem-less reporter probe configurations. Certain reporter probes are singly-labeled, while other reporter probes are doubly-labeled. Dual probe systems that comprise FRET between adjacently hybridized probes are also within the intended scope of the term reporter probe.

In certain embodiments, a reporter probe comprises a reporter group, a quencher (including without limitation dark quenchers and fluorescent quenchers), an affinity tag, a hybridization tag, a hybridization tag complement, or combinations thereof. In certain embodiments, a reporter probe comprising a hybridization tag complement anneals with the corresponding hybridization tag, a member of a multi-component reporter group binds to a reporter probe comprising the corresponding member of the multi-component reporter group, or combinations thereof. Exemplary reporter probes include TaqMan® probes; Scorpion probes (also referred to as scorpion primers); LuX™ primers; FRET primers; Eclipse probes; molecular beacons, including but not limited to FRET-based molecular beacons, multicolor molecular beacons, aptamer beacons, PNA beacons, and antibody beacons; reporter group-labeled PNA clamps, reporter group-labeled PNA openers, reporter group-labeled LNA probes, and probes comprising nanocrystals, metallic nanoparticles and similar hybrid probes (see, e.g., Dubertret et al., Nature Biotech. 19:365-70, 2001; Zelphati et al., BioTechniques 28:304-15, 2000). In certain embodiments, reporter probes further comprise groove binders including but not limited to TaqMan®MGB probes and TaqMan®MGB-NFQ probes (Applied Biosystems). In certain embodiments, reporter probes further comprise spanning or bridging oligonucleotides, and enhancer probes, for example but not limited to LNA-enhancer probes (see, e.g., Jacobsen et al., Nucl. Acid Res., 30(19):e100, 2002). In certain embodiments, reporter probe detection comprises fluorescence polarization detection (see, e.g., Simeonov and Nikiforov, Nucl. Acids Res. 30:e91, 2002).

As used herein, the terms antibody and antibodies are used in a broad sense, including not only intact antibody molecules, for example but not limited to immunoglobulin A, immunoglobulin G and immunoglobulin M, but also any immunoreactive component(s) of an antibody molecule that immunospecifically bind to an epitope. Such immunoreactive components include but are not limited to, FAb fragments, FAb′ fragments, FAb′2 fragments, single chain antibody fragments (scFv), miniantibodies, diabodies, crosslinked antibody fragments, Affibody® molecules, and the like. Immunoreactive products derived using antibody engineering or protein engineering techniques are also expressly within the meaning of the term antibodies. Detailed descriptions of antibody or protein engineering, including relevant protocols, can be found in, among other places, J. Maynard and G. Georgiou, Ann. Rev. Biomed. Eng. 2:339-76 (2000); Antibody Engineering, R. Kontermann and S. Dübel, eds., Springer Lab Manual, Springer Verlag (2001); A. Wörn and A. Plückthun, J. Mol. Biol. 305:989-1010 (2001); J. McCafferty et al., Nature 348:552-54 (1990); Müller et al., FEBS Letter, 432:45-9 (1998); A. Plückthun and P. Pack, Immunotechnology, 3:83-105 (1997); U.S. Pat. No. 5,831,012; and S. Paul, Antibody Engineering Protocols, Humana Press (1995).

Aptamers include nucleic acid aptamers (i.e., single-stranded DNA molecules or single-stranded RNA molecules) and peptide aptamers. Aptamers bind target molecules in a highly specific, conformation-dependent manner, typically with very high affinity, although aptamers with lower binding affinity can be selected if desired. Aptamers have been shown to distinguish between targets based on very small structural differences such as the presence or absence of a methyl or hydroxyl group and certain aptamers can distinguish between D- and L-enantiomers. Aptamers have been obtained that bind small molecular targets, including drugs, metal ions, and organic dyes, peptides, biotin, and proteins, including but not limited to streptavidin, VEGF, and viral proteins. Aptamers have been shown to retain functional activity after biotinylation, fluorescein labeling, and when attached to glass surfaces and microspheres.

Nucleic acid aptamers, including spiegelmers, are identified by an in vitro selection process known as systematic evolution of ligands by exponential amplification (SELEX). In the SELEX process very large combinatorial libraries of oligonucleotides, for example 10¹⁴ to 10¹⁵ individual sequences, often as large as 60-100 nucleotides long, are routinely screened by an iterative process of in vitro selection and amplification. Most targets are affinity enriched within 8-15 cycles and the process has been automated allowing for faster aptamer isolation. Peptide aptamers are typically identified by several different protein engineering techniques known in the art, including but not limited to, phage display, ribosome display, mRNA display, selectively infected phage technology (SIP), and the like. The skilled artisan will understand that nucleic acid aptamers and peptide aptamers can be obtained following conventional procedures and without undue experimentation. Detailed descriptions of aptamers, including relevant protocols, can be found in, among other places, L. Gold, J. Biol. Chem., 270(23):13581-84 (1995); L. Gold et al., Ann. Rev. Biochem. 64:763-97 (1995); S. Jayashena, Clin. Chem., 45:1628-50 (1999); Phage Display: A Laboratory Manual, C. Barbas, D. Burton, J. Scott, and G. Silverman, eds., Cold Spring Harbor Laboratory Press (2001); A. Plückthun et al., Adv. Protein Chem. 55:367-403 (2000); and Protein-Protein Interactions, A Molecular Cloning Manual, E. Golemis, ed., Cold Spring Harbor Press (2001).

III. Certain Exemplary Techniques

A target sequence according to the present teachings may be derived from any living, or once living, organism, including but not limited to, prokaryotes, archaea, viruses, and eukaryotes. The target nucleic acid may originate from the nucleus, typically genomic DNA, or may be extranuclear, e.g., plasmid, mitochondrial, viral, etc. The skilled artisan appreciates that genomic DNA includes not only full length material, but also fragments generated by any number of means, for example but not limited to, enzyme digestion, sonication, shear force, and the like. In certain embodiments, the target sequence may be present in a double-stranded or single-stranded form.

A variety of methods are available for obtaining a target sequence for use with the methods and kits of the present teachings. When the target sequences are obtained from a biological matrix, certain isolation techniques are typically employed, including without limitation, (1) organic extraction followed by ethanol precipitation, e.g., using a phenol/chloroform organic reagent (see, e.g., Ausubel et al., particularly Volume 1, Chapter 2, Section I), in certain embodiments, using an automated DNA extractor, e.g., the Model 341 DNA Extractor (Applied Biosystems); (2) stationary phase adsorption methods (see, e.g., U.S. Pat. No. 5,234,809; Walsh et al., BioTechniques 10(4): 506-513 (1991)); and (3) salt-induced DNA precipitation methods (see, e.g., Miller et al., Nucl. Acids Res. 16(3): 9-10, 1988), such precipitation methods being typically referred to as “salting-out” methods. In certain embodiments, the above isolation methods may be preceded by an enzyme digestion step to help eliminate unwanted protein from the sample, e.g., digestion with proteinase K, or other like proteases. See, e.g., U.S. patent application Ser. No. 09/724,613; see also, U.S. patent application Ser. Nos. 10/618,493 and 10/780,963; and U.S. Provisional Patent Application Ser. Nos. 60/499,082 and 60/523,056.

In certain embodiments, nucleic acids in a sample may be subjected to restriction enzyme cleavage and the resulting restriction fragments may be employed as target sequences. Different target sequences may be different portions of a single contiguous nucleic acid or may be on different nucleic acids. Different target sequences of a single contiguous nucleic acid may or may not overlap.

In certain embodiments, at least some of the target sequences are bisulfite modified, i.e., converted, prior to probe binding. Under appropriate conditions, bisulfite treatment converts unmethylated cytosine to uracil (U), while methylated cytosines are not converted (i.e., C=>U; 5mC=5mC). In certain embodiments, converted target sequences are amplified, thus the U nucleotides in the converted target sequences become T nucleotides in the amplified converted nucleic acid sequences (i.e., U=>T). Thus, in addition to the original complement of G, A, and T nucleotides, unconverted target sequences can comprise C nucleotides including 5-methylcytosine (5mC) nucleotides, while converted sequences comprise U nucleotides and 5mC nucleotides, and amplified converted sequences comprise C nucleotides and additional T nucleotides. Thus, when unconverted target sequences are employed in the current teachings, the target nucleotide is typically a C or 5mC and the complementary nucleotide (i.e., the pivotal complement) is a “G” nucleotide. However, when converted target sequences are employed in the current teachings, in certain embodiments, the pivotal complement can be a G or an A nucleotide, depending on whether the original target nucleotide was methylated or not.

For example, but without limitation, to interrogate a target nucleotide of unknown methylation in the context of converted DNA, a first cleavage probe set comprising two or more probe pairs can be used. In one exemplary embodiment, depicted in FIG. 1A, one probe pair includes a first cleavage probe comprising a first target region-binding portion with a G nucleotide on its 3′-end (shown as 1 G) and the corresponding second cleavage probe comprises a flap portion and a second target region-binding portion with a G nucleotide as its pivotal complement (shown as 2G). The related probe pair of this exemplary first cleavage probe set includes a first cleavage probe comprising a first target region-binding portion with an A nucleotide on its 3′-end (shown as 1A) and a corresponding second cleavage probe comprising a flap portion, a second target region-binding portion, and an A nucleotide as its pivotal complement (shown as 2A). Thus, the first probe pair in this illustrative first cleavage probe set is designed to interrogate the unconverted C target nucleotide (i.e., 5-methylcytosine) while the second probe pair in this example is designed to interrogate the converted target nucleotide U. Since the first target region and the second target region overlap by at least one nucleotide, each first cleavage probe first target region-binding portion overlaps the second target region-binding portion of the corresponding second cleavage probe by at least one nucleotide. Hence, when both the first and second cleavage probes are hybridized to their corresponding target regions, they create a suitable reaction substrate for a cleaving enzyme. The first target region-binding portions of alternate first cleavage probes of probe sets comprising two or more related probe pairs can be co-extensive, or the first target region-binding portion of one first cleavage probe may be a subset of the first target region-binding portion of the alternate cleavage probe of that probe set. Likewise, second target region-binding portions of alternate second cleavage probes of probe sets comprising two or more related probe pairs can be co-extensive, or the second target region-binding portion of one second cleavage probe may be a subset of the second target region-binding portion of an alternate second cleavage probe of that probe set.

In another illustrative embodiment, two or more coupled cleavage-ligation methylation detection assays are performed in parallel, or essentially in parallel, using the same cleavage probe set(s) with corresponding converted and unconverted target sequences (see, e.g., FIG. 3). By combining the exemplary 1G-2G probe set, the same cleaving enzyme, and the same ligation agent with the converted targets (“Tube 1”) and with the unconverted targets (“Tube 2”) under appropriate reaction conditions, the 1LP-G ligation yield for the two parallel reactions or the C_{T} for the two parallel reactions can be obtained, depending on the detecting and analyzing techniques employed. By comparing the two 1LP-G ligation yields, the corresponding 1LP-G ligation ratio, or the ΔC_{T}, the degree of target nucleotide methylation can be determined. Those in the art understand that a multiplicity of target nucleotides can be interrogated in such assays and they can be performed using a real-time instrument.

In certain embodiments, target sequence modifying agents other than sodium bisulfite may be used. In certain embodiments, the modifying agent need not catalyze deamination reactions and the converted nucleotide need not be uracil or thymine. Certain embodiments may employ any agent that is capable of selectively converting either methylated nucleotides or unmethylated nucleotides to another nucleotide.

According to the disclosed methods, modified or unmodified target sequences can be reacted with a first cleavage probe set under conditions effective to form a first hybridization complex (see, e.g., FIGS. 1A, 2, and 3). In the presence of a cleaving enzyme and under appropriate reaction conditions, the flap sequence of the annealed second cleavage probe of the first hybridization complex is cleaved and a second hybridization complex is formed, comprising the target sequence, an annealed first cleavage probe, and the hybridized fragment of the second cleavage probe (e.g., 2A* of FIG. 1A).

According to the disclosed methods, cleaving enzymes cleave flap portions from certain hybridization complexes, such as first hybridization complexes or fourth hybridization complexes (see, e.g., FIGS. 1A and 1B). Exemplary cleaving enzymes for use in the disclosed methods and kits include without limitation, E. coli DNA polymerase I, Thermus aquaticus DNA polymerase I, Thermus thermophilus DNA polymerase I, mammalian FEN-1, Archaeoglobus fulgidus FEN-1, Methanococcus jannaschii FEN-1, Pyrococcus furiosus FEN-1, Methanobacterium thermoautotrophicum FEN-1, Thermus thermophilus FEN-1, Cleavase® (enzymes (Third Wave, Inc., Madison, Wis.), Saccharomyces cerevisiae RTH1, S. cerevisiae RAD27 Schizosaccharomyces pombe rad2, bacteriophage T5 5′-3′ exonuclease, Pyroccus horikoshii FEN-1, human exonuclease 1, calf thymus 5′-3′ exonuclease, including homologs thereof in eubacteria, eukaryotes, and archaea, such as members of the class II family of structure-specific enzymes, as well as enzymatically active mutants or variants thereof. Those in the art understand that appropriate conditions for cleaving enzyme reactions are either known or can be readily determined using methods known in the art (see, e.g., Kaiser et al., J. Biol. Chem. 274:21387-94, 1999). Descriptions of cleaving enzymes can be found in, among other places, Lyamichev et al., Science 260:778-83, 1993; Eis et al., Nat. Biotechnol. 19:673-76, 2001; Shen et al., Trends in Bio. Sci. 23:171-73, 1998; Kaiser et al. J. Biol. Chem. 274:21387-94, 1999; Ma et al., J. Biol. Chem. 275:24693-700, 2000; Allawi et al., J. Mol. Biol. 328:537-54, 2003; Sharma et al., J. Biol. Chem. 278:23487-96, 2003; and Feng et al., Nat. Struct. Mol. Biol. 11:450-56, 2004.

According to certain disclosed methods, ligation of the annealed first cleavage probe and the annealed fragment of the second cleavage probe of the second and fifth hybridization complexes of the disclosed methods occurs under appropriate reaction conditions in the presence of a ligation agent to generate first and second ligation products as appropriate (see, e.g., FIGS. 1A and 1B). In certain embodiments, the third hybridization complex is denatured and the first ligation product and the target nucleic acid are released. In certain embodiments, when the sixth hybridization complex is denatured the first and second ligation products are released. In certain embodiments, the target sequence, the first ligation product, the second ligation product, or combinations thereof, are cycled through additional coupled cleavage-ligation reactions. In certain embodiments, the first or second ligation products or their surrogates are detected to determine the degree to which the target nucleotide is methylated. According to certain methods, a ligation product or a ligation product surrogate are separated before, or as part of, the determining process.

In certain embodiments, a first ligation product or a second ligation product are combined with a primer pair and are amplified, typically using the polymerase chain reaction (PCR), to generate amplified ligation products. In certain embodiments, a primer pair comprises a universal primer, i.e., a primer that is designed to hybridize with at least two different ligation product species. In certain embodiments, all of the primers are universal primers. In certain embodiments, an amplified ligation product comprises the complement of the full-length ligation product. In certain embodiments, an amplified ligation product does not comprise the complement of the full-length ligation product.

Certain embodiments of the disclosed methods and kits comprise a ligating step, a step for generating a ligation product, or a ligation means. Ligation, as that term is used herein, comprises any enzymatic or non-enzymatic technique wherein an inter-nucleotide linkage is formed between the opposing ends of nucleic acid sequences that are adjacently hybridized to a template, provided that those opposing ends are suitable for ligation. The inter-nucleotide linkage can include, but is not limited to, phosphodiester bond formation. Such bond formation can include, without limitation, those created enzymatically by a DNA ligase or a RNA ligase, for example but not limited to, T4 DNA ligase, T4 RNA ligase, Thermus thermophilus (Tth) ligase, Thermus aquaticus (Taq) DNA ligase, Thermus species AK16D ligase, Archaeoglobus fulgidus (Afu) ligase, or Pyrococcus furiosus (Pfu) ligase. Other inter-nucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages, typically generated using non-enzymatic ligation means, such as a chemical agent or photoligation.

Ligation generally comprises a cycle of ligation, i.e., the sequential procedures of: (1) hybridizing the sequence-specific portions of a first probe and a corresponding second probe, that are suitable for ligation, to their corresponding target regions or ligation product regions; (2) ligating (a) the 3′-end of the first cleavage probe with the 5′-end of the second cleavage probe fragment following a cleavage reaction, or (b) the 3′-end of the first ligation probe with the 5′-end of the corresponding second ligation probe; (3) and denaturing the nucleic acid duplex to release the ligation product from the corresponding hybridization complex (see, e.g., FIGS. 1 and 2). The ligation cycle may or may not be repeated, for example, without limitation, by thermocycling the coupled cleavage-ligation reaction or the ligation reaction to amplify the ligation product, either linearly or exponentially, depending on the assay. See also, U.S. patent application Ser. No. ______, entitled “Methods, Reaction Mixtures, and Kits for Ligating Polynucleotides”, by Andersen et al., filed Jun. 30, 2004.

Also within the scope of the current teachings are ligation techniques such as gap-filling ligation, including, without limitation, gap-filling OLA and LCR, bridging oligonucleotide ligation, and correction ligation. Descriptions of these techniques can be found in, among other places, U.S. Pat. No. 5,185,243, published European Patent Applications EP 320308 and EP 439182, PCT Publication Nos. WO 90/01069 and WO 01/57268, and Abravaya et al., Nucl. Acids Res. 23:675-82, 1995.

Certain embodiments of the disclosed methods and kits comprise a step for amplifying, a step for gap-filling, a step for extending a first cleavage probe, or an amplification means. Amplification according to the present teachings encompasses any means by which at least a part of a ligation product, at least part of a ligation product surrogate, or at least a part of a ligation product and at least part of a ligation product surrogate, is reproduced, typically in a template-dependent manner, including without limitation, a broad range of techniques for amplifying nucleic acid sequences, either linearly or exponentially. Exemplary means for performing an amplifying step include the polymerase chain reaction (PCR), primer extension, strand displacement amplification (SDA), multiple displacement amplification (MDA), nucleic acid strand-based amplification (NASBA), rolling circle amplification (RCA), transcription-mediated amplification (TMA), transcription, and the like, including multiplex versions or combinations thereof. Descriptions of such techniques can be found in, among other places, Sambrook and Russell; Sambrook et al.; Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); Rapley; U.S. Pat. Nos. 6,027,998 and 6,511,810; PCT Publication Nos. WO 97/31256 and WO 01/92579; Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000).

In certain embodiments, amplification comprises a cycle of the sequential steps of: hybridizing a primer with complementary or substantially complementary sequences in a ligation product, a ligation product surrogate, or a ligation product and a ligation product surrogate; synthesizing a strand of nucleotides in a template-dependent manner using a polymerase; and denaturing the newly-formed nucleic acid duplex to separate the strands. The cycle may or may not be repeated, as desired. Amplification can comprise thermocycling or can be performed isothermally. In certain embodiments, newly-formed nucleic acid duplexes are not initially denatured, but are used in their double-stranded form in one or more subsequent steps and either or both strands can, but need not, serve as ligation product surrogates. In certain embodiments, single-stranded amplicons are generated and can, but need not, serve as ligation product surrogates.

Primer extension is an amplifying technique that comprises elongating a probe or a primer that is annealed to a template in the 5′=>3′ direction using an amplifying means such as a polymerase. According to certain embodiments, with appropriate buffers, salts, pH, temperature, and nucleotide triphosphates, including analogs thereof, i.e., under appropriate amplification reaction conditions, a polymerase incorporates nucleotides complementary to the template strand starting at the 3′-end of an annealed probe or primer, to generate a complementary strand. In certain embodiments, primer extension can be used to fill a gap between two probes of a probe set that are hybridized to regions of a target sequence or a ligation product, so that the two probes can be ligated together. In certain embodiments, the polymerase used for primer extension lacks or substantially lacks 5′-exonuclease activity.

In certain embodiments, the disclosed methods and kits comprise a step for digesting or a digestion means, for example but not limited to enzymatic and chemical means for digesting at least part of a probe, at least part of a ligation product, at least part of an amplified ligation product, or combinations thereof. Exemplary enzymatic means for performing a digestion step include without limitation nucleases, for example but not limited to, endonucleases and exonucleases, such as BAL-31 nuclease, mung bean nuclease, exonuclease I, exonuclease III, λ exonuclease, T7 exonuclease, exonuclease T, recJ, uracil-N-glycosylase, and RNase H; restriction enzymes; and the like, including enzymatically active variants or mutants thereof. An alkaline hydrolysis step for digesting the RNA portion of an RNA-DNA hybrid or RNA:DNA duplex is one example of chemical digestion means.

The skilled artisan will understand that any of a number of nucleases, polymerases, cleaving enzymes, and ligases could be used in the disclosed methods and kits, including without limitation, those isolated from thermostable or hyperthermostable prokaryotic, eukaryotic, or archaeal organisms. The skilled artisan will also understand that enzymes such as “structure-specific nuclease”, “flap endonuclease”, “FEN-1”, “ligase”, “nuclease”, “polymerase”, and so forth, include not only naturally occurring enzymes, but also recombinant enzymes; and enzymatically active fragments, cleavage products, mutants, or variants of such enzymes, for example but not limited to Klenow fragment, Stoffel fragment, Taq FS (Applied Biosystems), 9° Nₘ™ DNA Polymerase (New England BioLabs, Beverly, Mass.), and mutant enzymes (including without limitation, naturally-occurring and man-made mutants), described in Luo and Barany, Nucl. Acids Res. 24:3079-3085 (1996), Eis et al., Nature Biotechnol. 19:673-76 (2001), and U.S. Pat. Nos. 6,265,193 and 6,576,453. Reversibly modified nucleases, ligases, and polymerases, for example but not limited to those described in U.S. Pat. No. 5,773,258, are also within the scope of the disclosed teachings. Those in the art will understand that any protein with the desired enzymatic activity, be it cleaving, ligating, amplifying, or digesting, can be used in the disclosed methods and kits. Descriptions of nucleases, ligases, and polymerases can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Ausubel et al.; Lyamichev et al., Science 260:778-783, 1993; Allawi et al., J. Mol. Biol. 328:537-54, 2003; Kaiser et al., J. Biol. Chem. 274:21387-94, 1999; Hosfield et al., J. Biol. Chem. 273:27154-61, 1998; Matsui et al., J. Biol. Chem. 274:18297-309, 1999; and Murante et al., J. Biol. Chem. 269:1191-96, 1994.

Certain embodiments of the disclosed methods and kits comprise separating (either as a separate step or as part of a step for determining) or a separation means. Separating comprises any process that removes at least some unreacted components or at least some reagents from a cleaved flap, a ligation product, a ligation product surrogate, or combinations thereof. In certain embodiments, a cleaved flap, a ligation product, an amplified ligation product, a digested ligation product, a digested amplified ligation product, or combinations thereof, are separated from unreacted components and reagents, including without limitation, unreacted molecular species present in the sample, cleaving enzymes, ligation reagents, and amplification reagents, for example, but not limited to, cleavage probes, ligation probes, primers, enzymes, co-factors, unbound sample components, nucleotides, and the like. In certain embodiments, a cleaved flap is separated from a hybridization complex, a first ligation product is separated from a target sequence, a first ligation product is separated from a second ligation product, or combinations thereof. The skilled artisan will appreciate that a number of well-known separation means can be used in the methods and kits disclosed herein.

Exemplary means/techniques for performing a separation step include gel electrophoresis, for example but not limited to, isoelectric focusing and capillary electrophoresis; dielectrophoresis; flow cytometry, including but not limited to fluorescence-activated sorting techniques using beads, microspheres, or the like; liquid chromatography, including without limitation, HPLC, FPLC, size exclusion (gel filtration) chromatography, affinity chromatography, ion exchange chromatography, hydrophobic interaction chromatography, immunoaffinity chromatography, and reverse phase chromatography; affinity tag binding, such as biotin-avidin, biotin-streptavidin, maltose-maltose binding protein (MBP), and calcium-calcium binding peptide; aptamer-target binding; hybridization tag-hybridization tag complement annealing; mass spectrometry, including without limitation MALDI-TOF, MALDI-TOF-TOF, tandem mass spec (MS-MS), LC-MS, and LC-MS/MS; a microfluidic device; and the like. Detailed discussion of separation techniques can be found in, among other places, Rapley; Sambrook et al.; Sambrook and Russell; Ausubel et al.; Handbook of Fluorescent Probes and Research Products, 9^{th} ed., R. Haugland, Molecular Probes, Inc., 2002 (“Molecular Probes Handbook”); Pierce Applications Handbook; Capillary Electrophoresis: Theory and Practice, P. Grossman and J. Colburn, eds., Academic Press, 1992; PCT Publication No. WO 01/92579; and M. Ladisch, Bioseparations Engineering: Principles, Practice, and Economics, John Wiley & Sons, 2001.

In certain embodiments, a separating step comprises binding or annealing a cleavage probe, a target sequence, a hybridization complex, a ligation product or its surrogate, or combinations thereof, to a Substrate, for example but not limited to binding a biotinylated ligation product to a streptavidin-coated Substrate or binding a ligation product comprising a hybridization tag to a Substrate comprising a hybridization tag complement at a unique address on the Substrate. Suitable Substrates include but are not limited to: microarrays, including fixed arrays and bead arrays; appropriately treated or coated reaction vessels and surfaces; beads, for example but not limited to magnetic beads, paramagnetic beads, latex beads, metallic beads, polymer beads, dye-impregnated beads, and coated beads; optically identifiable micro-cylinders; biosensors comprising transducers; and the like (see, e.g., Tong et al., Nat. Biotech. 19:756-59 (2001); Gerry et al., J. Mol. Biol. 292:251-62 (1999); Srisawat et al., Nucl. Acids Res. 29:e4 (2001); Han et al., Nat. Biotech. 19:631-35, 2001; and Stears et al., Nat. Med. 9:140-45, including supplements, 2003). Those in the art will appreciate that the shape and composition of the Substrate is generally not limiting.

In certain embodiments, a cleaved flap, a ligation product, a ligation product surrogate, or combinations thereof are resolved (separated) by liquid chromatography. Exemplary stationary phase chromatography media for use in the teachings herein include reversed-phase media (e.g., C-18 or C-8 solid phases), ion-exchange media (particularly anion-exchange media), and hydrophobic interaction media. In certain embodiments, a cleaved flap, a ligation product, a ligation product surrogate, or combinations thereof can be separated by micellar electrokinetic capillary chromatography (MECC).

Reversed-phase chromatography is carried out using an isocratic, or more typically, a linear, curved, or stepped solvent gradient, wherein the level of a nonpolar solvent such as acetonitrile or isopropanol in aqueous solvent is increased during a chromatographic run, causing analytes to elute sequentially according to affinity of each analyte for the solid phase. For separating polynucleotides, including ligation products and at least some ligation product surrogates, an ion-pairing agent (e.g., a tetra-alkylammonium) is typically included in the solvent to mask the charge of phosphate.

The mobility of cleaved flaps, ligation products, and at least some ligation product surrogates can be varied by using mobility modifiers comprising polymer chains that alter the affinity of the element to which it is attached for the solid, or stationary phase. Thus, with reversed phase chromatography, an increased affinity of the cleaved flaps, the ligation products and at least some ligation product surrogates for the stationary phase can be attained by adding a moderately hydrophobic tail (e.g., PEO-containing polymers, short polypeptides, and the like) to the mobility modifier. Longer tails impart greater affinity for the solid phase, and thus require higher non-polar solvent concentration for the ligation products or ligation product surrogates to be eluted (and a longer elution time).

In certain embodiments, a cleaved flap, a ligation product, a ligation product surrogate, or combinations thereof, are resolved by electrophoresis in a sieving or non-sieving matrix. In certain embodiments, the electrophoretic separation is carried out in a capillary tube by capillary electrophoresis, including without limitation, microcapillaries and nanocapillaries (see, e.g., Capillary Electrophoresis: Theory and Practice, Grossman and Colburn eds., Academic Press, 1992). Exemplary sieving matrices for use in the disclosed teachings include covalently crosslinked matrices, such as polyacrylamide covalently crosslinked with bis-acrylamide; gel matrices formed with linear polymers (see, e.g., U.S. Pat. No. 5,552,028); and gel-free sieving media (see, e.g., U.S. Pat. No. 5,624,800; Hubert and Slater, Electrophoresis, 16: 2137-2142, 1995; Mayer et al., Analytical Chemistry, 66(10):1777-1780, 1994). The electrophoresis medium may contain a nucleic acid denaturant, such as 7M formamide, for maintaining polynucleotides in single stranded form. Suitable capillary electrophoresis instrumentation are commercially available, e.g., the ABI PRISM™ Genetic Analyzer series (Applied Biosystems).

In certain embodiments, a hybridization tag complement includes a hybridization enhancer, where, as used herein, the term “hybridization enhancer” means moieties that serve to enhance, stabilize, or otherwise positively influence hybridization between two polynucleotides, e.g. intercalators (see, e.g., U.S. Pat. No. 4,835,263), minor-groove binders (see, e.g., U.S. Pat. No. 5,801,155), and cross-linking functional groups. The hybridization enhancer may be attached to any portion of a mobility modifier, so long as it is attached to the mobility modifier is such a way as to allow interaction with the hybridization tag-hybridization tag complement duplex. In certain embodiments, a hybridization enhancer comprises a minor-groove binder, e.g., netropsin, distamycin, and the like.

The skilled artisan will appreciate that a cleaved flap, a ligation product, a ligation product surrogate, or combinations thereof can also be separated based on molecular weight and length or mobility by, for example, but without limitation, gel filtration, mass spectrometry, or HPLC, and detected using appropriate methods. In certain embodiments, a cleaved flap, a ligation product, a ligation product surrogate, or combinations thereof are separated using one or more of the following forces: gravity, electrical, centrifugal, hydraulic, pneumatic, or magnetism.

In certain embodiments, an affinity tag is used to separate the element to which it is bound, e.g., a cleaved flap, a ligation product, a ligation product surrogate, or combinations thereof, from a component of a coupled cleavage-ligation reaction composition, a ligation reaction composition, a digestion reaction composition, an amplified ligation reaction composition, or the like. In certain embodiments, an affinity tag is used to bind a ligation product, a ligation product surrogate, or combinations thereof to a Substrate, for example but not limited to binding a digoxygenin-labeled ligation product to a Substrate comprising anti-digoxygenin antibody. In certain embodiments, an aptamer is used to bind a ligation product, a ligation product surrogate, or combinations thereof, to a Substrate (see, e.g., Srisawat and Engelke, RNA 7:632-641 (2001); Holeman et al., Fold Des. 3:423-31 (1998); Srisawat et al., Nucl. Acid Res. 29(2):e4, 2001). In certain embodiments, one strand of a hybridization complex comprises a biotin affinity tag and the hybridization complex is bound to a streptavidin-coated Substrate. In certain embodiments, the Substrate-bound hybridization complex is denatured and the non-biotinylated strand is released from the Substrate. In certain embodiments, the released strand or its surrogate is subsequently detected.

In certain embodiments, a hybridization tag, a hybridization tag complement, or a hybridization tag and a hybridization tag complement, is used to separate the element to which it is bound from a ligation reaction composition, a digestion reaction composition, an amplified ligation reaction composition, or the like. In certain embodiments, hybridization tags are used to attach a ligation product, a ligation product surrogate, or combinations thereof, to a Substrate. In certain embodiments, a ligation product, a ligation product surrogate, or combinations thereof, comprise the same hybridization tag. For example but not limited to, separating a multiplicity of different element:hybridization tag species using the same hybridization tag complement, tethering a multiplicity of different element:hybridization tag species to a Substrate comprising the same hybridization tag complement.

In certain embodiments, separation comprises binding a ligation product or a ligation product surrogate to a Substrate, either directly or indirectly; for example but not limited to, indirectly binding a ligation product to a glass Substrate, wherein the ligation product comprises an affinity tag such as biotin, and the Substrate comprises a corresponding affinity tag, such as a streptavidin, avidin, CaptAvidin, or NeutrAvidin; or vice versa. The skilled artisan will understand that certain methods comprise at least two different separations, for example a first bulk separation and a second separation wherein, for example, a ligation product comprising an affinity tag is attached to a Substrate comprising a corresponding affinity tag. For example, but without limitation, separating a ligation product comprising a DNP affinity tag by capillary electrophoresis and then tethering the DNP-ligation product indirectly to a particular address on a Substrate comprising anti-DNP antibody; separating a ligation product comprising an hybridization tag by RP-HPLC and then indirectly binding the ligation product to a glass, mica, or silicon Substrate comprising the corresponding hybridization tag complement; or binding a hybridization complex comprising a biotinylated ligation product and a second ligation product to a streptavidin-coated Substrate to separate it from unbound components of a coupled cleavage-ligation reaction composition, denaturing the hybridization complex to release the second ligation product, then subjecting the released second ligation product or its surrogate to capillary electrophoresis.

In certain embodiments, a Substrate is derivatized or coated to enhance the binding of an affinity tag, a ligation product, a hybridization tag complement, a cleaved flap, or combinations thereof. Exemplary Substrate treatments and coatings include poly-lysine coating; aldehyde treatment; amine treatment; epoxide treatment; sulphur-based treatment (e.g., isothiocyanate, mercapto, thiol); coating with avidin, streptavidin, biotin, or derivatives thereof; and the like. Detailed descriptions of derivatization techniques and procedures to enhance capture moiety binding can be found in, among other places, Microarray Analysis; G. MacBeath and S. Schreiber, Science 289:1760-63 (2000); A, Talapatra, R. Rouse, and G. Hardiman, Proteogenomics 3:1-10 (2002); Microarray Methods and Applications-Nuts and Bolts, G. Hardiman, ed., DNA Press (2003); B. Houseman and M. Mrksich, Trends in Biochemistry 20:279-81 (2002); S. Carmichael et al., A Simple Test Method for Covalent Binding Microarray Surfaces, NoAb BioDiscoveries Microarray Technical Note #010516SC; P. Galvin, An introduction to analysis of differential gene expression using DNA microarrays, The European Working Group on CTFR Expression (Apr. 2, 2003); and Zhu et al., Curr. Opin. Chem. Biol. 7:55-63 (2003). Pretreated Substrates and derivatization reagents and kits are commercially available from several sources, including CEL Associates, Pearland Tex.; Molecular Probes, Eugene Oreg.; Quantifoil MicroTools GmbH, Jena Germany; Xenopore Corp., Hawthorne, N.J.; NoAb BioDiscoveries, Mississauga, Ontario, Canada; TeleChem International, Sunnyvale, Calif.; CLONTECH Laboratories, Inc., Palo Alto Calif.; and Accelr8 Technology Corp., Denver, Colo. In certain embodiments, the Substrate-bound capture moiety comprises an amino acid, for example but not limited to, antibodies, peptide aptamers, peptides, avidin, streptavidin, biotin, and the like. In certain embodiments, the Substrate-bound capture moiety comprises a nucleotide, for example but not limited to, hybridization tag complements, nucleic acid aptamers, and chimeric oligomers further comprising PNAs, pcPNAs, LNAs, and the like.

In certain embodiments, a first cleavage probe, a second cleavage probe, a first ligation probe, a second ligation probe, a target sequence, a converted target sequence, a ligation product, or combinations thereof, are bound directly or indirectly to a Substrate. In certain embodiments, a Substrate comprises a bound hybridization complex. In certain embodiments, a hybridization complex is formed on a Substrate, a cleavage reaction occurs on a Substrate-bound hybridization complex, a ligation reaction occurs on a Substrate-bound hybridization complex, or combinations thereof. In certain embodiments, a separating step and a determining step comprise a Substrate, wherein the Substrate can be the same or different. For example, a first Substrate for bulk separation, and a second Substrate for detecting and quantifying the ligation products, ligation product surrogates, cleaved flaps, hybridization tag complements, or combinations thereof.

The disclosed methods and kits comprise a step for determining the degree of target nucleotide methylation or a determining means. Such determining comprises any means by which the methylation state of a target nucleotide is identified or inferred, including but not limited to evaluating the degree of methylation of a target nucleotide. In certain embodiments, determining comprises quantifying the cleaved flaps, ligation products, ligation product surrogates, or combinations thereof, that are detected using, for example but not limited to graphically displaying the quantified cleaved flaps, ligation products, ligation product surrogates, ΔC_{T}, ΔΔC_{T}, or combinations thereof on a graph, monitor, electronic screen, magnetic media, scanner print-out, or other two- or three-dimensional display. Typically the peak height, the area under the peak, the signal intensity, or combinations thereof, of one or more detected reporter group on the ligation product or ligation product surrogate, or other quantifiable parameter of the ligation product or surrogate are measured and the amount of ligation product that was produced in a particular ligation assay is inferred. Generally, a quantified parameter for a ligation product, a ligation product surrogate, or combinations thereof, is compared to the same parameter(s) from a second ligation product, a second ligation product surrogate, or combinations thereof and a ratio of the two ligation products is obtained. In certain embodiments, the degree of target nucleotide methylation is determined by evaluating the ligation ratio of two ligation products, for example but not limited to, the ligation ratio of the ligation products obtained using converted target sequences with related probe pairs (see, e.g., FIG. 2).

By comparing the experimentally obtained ligation yield for a given cleaving enzyme-ligation agent combination for a probe sets or at least a probe pair of that probe set with a control ligation yield, for example but not limited to, relative and absolute standard curves, and ligation yields for certain “housekeeping” genes, the degree of methylation of a target nucleotide species can be determined. In certain embodiments, the ligation yield of related probe pairs are compared to determine the degree of target nucleotide methylation. Related probe pairs are two or more probe pairs that can each be used to interrogate the same target nucleotide, including without limitation, a first probe pair that can form a second hybridization complex with a converted target sequence when given target nucleotide is a methylated cytosine (but not a uracil) and a second probe pair that can form a second hybridization complex with the converted target sequence when the target nucleotide is uracil (but not methylated cytosine). Control ligation yields can be pre-determined, analyzed in one or more parallel coupled cleavage-ligation reactions, or determined subsequently.

In certain embodiments, determining the degree of methylation of a target nucleotide comprises comparing the amount of cleaved flaps, ligation products, ligation product surrogates, or combinations thereof, obtained using converted nucleic acid sequences with the corresponding amount of cleaved flaps, ligation products, ligation product surrogates, or combinations thereof, obtained using unconverted nucleic acid sequences, including without limitation, obtaining and evaluating the ligation ratio of a probe set using the same targets or the same probes using converted versus unconverted targets (see, e.g., FIG. 3). For example but not limited to, comparing (a) the amount of ligation product obtained using a particular first cleavage probe set and converted target sequences with (b) the amount of ligation product obtained with the same cleavage probe set and unconverted target sequences. In certain embodiments, the determining comprises visual, automated, or semi-automated comparison of peak heights, peak areas, signal intensity, ΔC_{T}, and the like. In certain embodiments, a determining step comprises using a computer algorithm, including without limitation, standard curve analysis.

By comparing the ligation ratio obtained from an unknown sample with control ratios or standard curves for the same target nucleotide and using the same probes and assay conditions, one can determine the methylation state of the target nucleotide. For example, consider an illustrative coupled cleavage-ligation assay with two possible ligation products from related probe pairs, e.g., LP1 and LP2. Assume in this illustration that the LP1:LP2 ratio for a particular unknown sample is 5:1 and the LP1:LP2 ratio obtained using a methylated control target sequence (e.g., a synthetic target in which all of the target nucleotides are 5-methylcytosine) was 5:1 and with a control target sequence wherein all of the target nucleotides are converted to uracil was 1:1. By comparing the ligation ratio obtained using the unknown sample with the two control samples, one can determine that all or substantially all of the target nucleotide in the unknown sample was fully methylated. When the ligation ratio obtained using the unknown sample is between 5:1 and 1:1 in this illustration, one can infer that the degree of target nucleotide methylation has an intermediate value that depends on those two control ratios. Using the standard curve for those probe pairs using the same reaction conditions, one can plot the experimentally obtained ligation ratio on the curve and determine the corresponding degree of methylation for the target nucleotide.

The generation and use of standard curves is well known to those in the art (see, e.g., Overholtzer et al., Proc. Natl. Acad. Sci. 100:11547-52, 2003; Simeonov and Nikiforov, Nucl. Acids Res. 30:e91, 2002; and Osiowy, J. Clin. Micro. 40:2566-71, 2002). Typically, a standard curve is generated by plotting experimentally obtained results for a particular set of reagents and under defined assay conditions on an X-Y graph or other coordinate system and then generating a curve, generally either manually or using one or more mathematical formula or algorithm, for example but not limited to graphing or line drawing software, linear regression analysis and similar mathematical calculations, computer algorithms, or the like. Once a standard curve have been generated for a given target nucleotide and a corresponding probe set or at least a probe pair of that probe set, experimentally-determined results obtained from test (unknown) samples using the same probes under the same assay conditions can be evaluated using the standard curve and the degree of target nucleotide methylation determined. The skilled artisan will appreciate that a “curve” can actually be a straight or substantially straight line or it can be curvilinear and assume a wide range of shapes.

In certain embodiments, a determining step comprises separating, detecting, and quantifying a ligation product parameter using an instrument, i.e., using an automated or semi-automated determining means that can, but need not, comprise a computer algorithm. In certain embodiments, the determining step is combined with or is a continuation of a separating step, for example but not limited to a capillary electrophoresis instrument comprising a fluorescent scanner and a graphing, recording, or readout component; a capillary electrophoresis instrument coupled with a mass spectrometer; a chromatography column coupled with an absorbance monitor or fluorescence scanner and a graph recorder, or with a mass spectrometer; or a microarray with a data recording device such as a scanner or CCD camera. Exemplary means for performing a determining step include capillary electrophoresis instruments, for example but not limited to, the ABI PRISM® 3100 Genetic Analyzer, ABI PRISM® 3100-Avant Genetic Analyzer, ABI PRISM® 3700 DNA Analyzer, ABI PRISM® 3730 DNA Analyzer, ABI PRISM® 3730xl DNA Analyzer (all from Applied Biosystems); the ABI PRISM® 7300 Real-Time PCR System; the ABI PRISM® 7700 Sequence Detection System; mass spectrometers; and microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:140-45, including supplements, 2003). Exemplary software for reporter group detection, data collection, and analysis includes GeneMapper™ Software, GeneScan® Analysis Software, and Genotyper® software (all from Applied Biosystems).

In certain embodiments, separating or determining comprises flow cytometry methods, including without limitation fluorescence-activated sorting (see, e.g., Vignali, J. Immunol. Methods 243:243-55, 2000). In certain embodiments, determining comprises: separating a plurality of cleaved flaps, ligation products, ligation product surrogates, or combinations thereof using a mobility-dependent analytical technique, such as capillary electrophoresis; monitoring the eluate using, for example but without limitation, a fluorescent scanner, to detect the separated ligation products or ligation product surrogates as they elute; and evaluating the fluorescent profile of the ligation products, ligation product surrogates, cleaved flaps, or combinations thereof, typically using detection and analysis software, such as an ABI PRISM® Genetic Analyzer using GeneScan® Analysis Software (both from Applied Biosystems). In certain embodiments, determining comprises a plate reader and an appropriate light source.

In certain embodiments, the cleaved flaps, ligation products, ligation product surrogates, or combinations thereof do not comprise reporter groups, but are detected and quantified based on their corresponding mass-to-charge ratios (m/z). In certain embodiments, a multiplicity of ligation products, ligation products surrogates, cleaved flaps, or combinations thereof, are separated by liquid chromatography or capillary electrophoresis, subjected to ESI, and detected by mass spectrometry. In certain embodiments, a multiplicity of ligation products, ligation product surrogates, cleaved flaps, or combinations thereof, are subjected to MALDI and detected by mass spectrometry.

In certain embodiments, a cleavage probe, a ligation product, a ligation product surrogate, a cleaved flap, or combinations thereof, are hybridized or attached to a Substrate, including without limitation, a microarray or a bead. In certain embodiments, a Substrate-bound ligation product, Substrate-bound ligation product surrogate, Substrate-bound cleaved flap, or combinations thereof, do not comprise an integrated reporter group, but are detected due to the hybridization of a labeled entity. Such labeled entity include without limitation, a labeled hybridization tag complement, a reporter probe such as a molecular beacon, a light-up probe, a labeled LNA probe, a labeled PNA probe, or the capture probe of the Substrate. In certain embodiments, the labeled entity comprises a fluorescent reporter group and quencher.

In certain embodiments, determining comprises detecting a reporter probe, the reporter group of a released hybridization tag complement or a part of a hybridization tag complement, a reporter group on a cleaved flap, or other indirect ligation product detection method. For example, without limitation, hybridizing a cleaved flap, ligation product, or ligation product surrogate to a labeled probe comprising a quencher, including without limitation, a molecular beacon, including stem-loop and stem-free beacons, a TaqMan® probe, or a microarray capture probe. In certain embodiments, the hybridization occurs in solution such as hybridizing a molecular beacon to a ligation product. In other embodiments, the cleaved flap, ligation product, ligation product surrogate, or the reporter probe is Substrate-bound and upon hybridization of the corresponding reporter probe, ligation product, ligation product surrogate, or cleaved flap, fluorescence is detected (see, e.g., EviArrays™ and EviProbes™, Evident Technologies). In certain embodiments, such hybridization events are simultaneously or near-simultaneously detected and quantified.

In certain embodiments, determining comprises detecting a single-stranded molecule, such as a cleaved flap, a ligation product, or a single-stranded ligation product surrogate. Such detecting can comprise, among other things, a reporter group that is integral to the single-stranded molecule being detected, such as a fluorescent reporter group that is incorporated into a probe; a reporter group on a molecule that hybridizes with the single-stranded molecule being detected, such as a hybridization tag complement or a molecular beacon, including without limitation, PNA beacons and LNA beacons, a TaqMan® probe, a scorpion primer, or a light-up probe. In certain embodiments, determining comprises detected a double-stranded molecule, including without limitation a third hybridization complex (comprising a target sequence and a first ligation product), a sixth hybridization complex (comprising a first ligation product and a second ligation product), an eighth hybridization product (comprising a first ligation product and a third ligation product), a tenth hybridization complex (comprising a second ligation product and a fourth ligation product), a double-stranded amplified ligation product (for example but not limited to, a first ligation product hybridized with its corresponding single-stranded amplicon or a double-stranded amplicon), or a double-stranded digested amplified ligation product. Typically such double-stranded molecules are detected by triplex formation or by local opening of the double-stranded molecule, using for example but without limitation, a PNA opener, a PNA clamp, and triplex forming oligonucleotides (TFOs), either reporter group-labeled or used in conjunction with a labeled entity such as a molecular beacon (see, e.g., Drewe et al., Mol. Cell. Probes 14:269-83, 2000; Zelphati et al., BioTechniques 28:304-15, 2000; Kuhn et al., J. Amer. Chem. Soc. 124:1097-1103, 2002; Knauert and Glazer, Hum. Mol. Genet. 10:2243-2251, 2001; Lohse et al., Bioconj. Chem. 8:503-09, 1997). In certain embodiments, a reporter probe-binding portion of a probe, a reporter probe-binding portion of a primer or a reporter probe-binding portion of a probe and a reporter probe-binding portion of a primer, comprises a homopurine stretch.

In certain embodiments, determining comprises measuring or quantifying the detectable signal of a reporter group. In certain embodiments, determining comprises measuring or quantifying the change in a detectable signal, typically due to the presence of a cleaved flap, a ligation product, a hybridization complex, an amplified ligation product, or the like. For example, but not limited to, an unhybridized reporter probe may emit a low level, but detectable signal that quantitatively increases when hybridized, including without limitation, certain molecular beacons, LNA probes, PNA probes, and light-up probes (see, e.g., Svanik et al., Analyt. Biochem. 281:26-35, 2000; Nikiforov and Jeong, Analyt. Biochem. 275:248-53, 1999; and Simeonov and Nikiforov, Nucl. Acids Res. 30:e91, 2002). In certain embodiments, detecting comprises measuring fluorescence polarization.

Those in the art understand that the separation or determining means employed are generally not limiting. Rather, a wide variety of separation and determining means, including without limitation detecting and analyzing means, are within the scope of the disclosed methods and kits.

In one illustrative protocol, the methylation status of a target nucleotide in the death-associated protein kinase (DAPK) promoter is determined as follows (see, e.g., FIG. 2 in part). A gDNA sample comprising the target sequence: 3′-TCGATCCCTCACTCACCCCCCGCGTCTAGGGAGGGTCCG-5′ (SEQ ID NO:1; target nucleotide shown underlined) is bisulfite modified using well known methods to generate the converted target sequence 3′-TUGATUUUTUAUTUAUUUUUUGYGTUTAGGGAGGGTUUG-5′ (SEQ ID NO:2), where Y represents C or U, depending on whether the target nucleotide is methylated or not. Two aliquots of the converted sample are analyzed in parallel by combining in separate wells of a 96-well microplate. One well includes an aliquot of the converted target sequences; a first cleavage probe pair comprising a first cleavage probe with the sequence 5′-AACTAAAAAATAAATAAAAAACA-3′ (SEQ ID NO:3) and a corresponding second cleavage probe with the sequence 5′-A*ACAAATCCCTCCCAAAC#-3′ (SEQ ID NO:4); a second cleavage probe pair comprising a first cleavage probe with the sequence 5′-GTTTGGGAGGGATTTGT-3′ (SEQ ID NO:5) and a corresponding second cleavage probe with the sequence 5′-C*TGTTTTTTTATTTATTTTTTAGTT#-3′ (SEQ ID NO:6), wherein the 5′ nucleotide of both second cleavage probes (shown as “A*” and “C*” respectively) comprise a FAM reporter group and the 3′ nucleotide of both second cleavage probes comprise a TAMRA reporter group (shown as “C#” and “T™” respectively); Thermus species AK16D ligase; a thermostable flap endonuclease; and reaction buffer (the “U-specific” assay). The second well includes: an aliquot of the converted target sequences; a different first cleavage probe pair comprising a first cleavage probe with the sequence 5′-AACTAAAAAATAAATAAAAAACG-3′ (SEQ ID NO:7) and a corresponding second cleavage probe with the sequence 5′-A*GCAAATCCCTCCCAAAC#-3′ (SEQ ID NO:8); a second cleavage probe pair comprising a first cleavage probe with the sequence 5′-GTTTGGGAGGGATTTGC-3′ (SEQ ID NO:9) and a corresponding second cleavage probe with the sequence 5′-C*CGTTTTTTATTTATTTTTTAGTT#-3′ (SEQ ID NO:10), nucleotide of both second cleavage probes (shown as “A*” and “C*” respectively) comprise a FAM reporter group and the 3′ nucleotide of both second cleavage probes comprise a TAMRA reporter group (shown as C# and T# respectively); Thermus species AK16D ligase; a thermostable flap endonuclease; and reaction buffer (the “C-specific” assay). The 96-well microplate is loaded in ABI 7700 Sequence Detection System, and cycled according to the manufacturer's instructions (modified as necessary for the coupled cleavage-ligation reaction). The C_{T} value for the “U-specific” ligation products and the “C-specific” ligation products are obtained and their ΔC_{T} calculated. Using this ΔC_{T} value, the degree of methylation of the exemplary DAPK promoter target nucleotide is determined. Those in the art will appreciate that additional target nucleotides in the DAPK promoter or other target sequences can be analyzed, either individually or in a multiplexed assay, using similar methodology, and the corresponding degree of methylation of the respective target nucleotides determined.

According to the present teachings, a step for interrogating a target nucleotide is performed using the disclosed cleavage probe sets; a step for generating a cleaved flap is performed using the disclosed cleaving enzymes; a step for generating a ligation product is performed using the disclosed ligation agents and ligation techniques with either (1) a cleavage probe set comprising a first cleavage probe and a fragment of second cleavage probe, or (2) the first and second probes of a ligation probe set; a step for generating an amplified ligation product, a step for gap-filling, or a step for extending a first cleavage probe, are performed using the disclosed amplifying means and amplification techniques; a step for generating a digested ligation product is performed using the disclosed nucleases, restriction enzymes, chemical digesting means, and digestion techniques; and a step for determining the degree of methylation of a target nucleotide is performed using a disclosed detecting technique comprising a disclosed quantifying technique, a disclosed analyzing technique, a disclosed separating technique, or combinations thereof.

IV. Certain Exemplary Kits

The instant teachings also provide kits designed to expedite performing the subject methods. Kits serve to expedite the performance of the disclosed methods by assembling two or more components required for carrying out the methods. Kits generally contain components in pre-measured unit amounts to minimize the need for measurements by end-users. Kits preferably include instructions for performing one or more of the disclosed methods. Typically, the kit components are optimized to operate in conjunction with one another.

Kits for determining the degree of methylation of a target nucleotide are provided. The disclosed kits may be used to generate a cleaved flap and a ligation product, typically in a coupled reaction (e.g., the reaction composition comprises target sequences, cleavage probes, a cleaving enzyme, and a ligation agent). In certain embodiments, the disclosed kits may also be used to generate an amplified ligation product, an amplified digested ligation product, a digested ligation product, or combinations thereof. In certain embodiments, the instant kits comprise a cleaving enzyme, a ligase, a polymerase, a nuclease, including enzymatically active mutants or variants of each of these four types of enzymes; a reporter group; a mobility modifier; an affinity tag; a hybridization tag; a cleavage probe set; a ligation probe set; a primer; or combinations thereof. In certain embodiments, kits comprise a means for cleaving, a means for ligating, a means for separating, a means for digesting, a detection means, an identifying means, or combinations thereof.

In certain embodiments the disclosed methods and kits further comprise an amplifying means, for example a polymerase, including, but not limited to a DNA polymerase, an RNA polymerase, a reverse transcriptase, or combinations thereof. Such polymerases provide a means for amplifying a nucleotide. Exemplary polymerases include DNA polymerase I, T4 DNA polymerase, SP6 RNA polymerase, T3 RNA polymerase, T7 RNA polymerase, AMV reverse transcriptase, M-MLV reverse transcriptase, and the like. In certain embodiments, a DNA polymerase lacks 5′=>3′ exonuclease activity, for example, but not limited to Klenow fragment of DNA polymerase, 9° Nₘ™ DNA polymerase, Vent_{R}® (exo⁻) DNA polymerase, Deep Vent_{R}® (exo⁻) DNA polymerase, Therminator™ DNA polymerase, and the like. In certain embodiments, a polymerase is thermostable. Exemplary thermostable polymerases include Taq polymerase, Tfl polymerase, Tth polymerase, Tli polymerase, Pfu polymerase, AmpliTaq Gold® polymerase, 9° Nₘ™ DNA polymerase, Vent_{R}® DNA polymerase, Deep Vent_{R}® DNA polymerase, UlTma polymerase, and the like.

In certain embodiments, the methods and kits disclosed herein comprise a polymerase, a ligation agent, a digestion agent, or combinations thereof. In certain embodiments, the methods disclosed herein comprise ligation reactions and can further comprise primer extension, including but not limited to “gap filling” reactions and the polymerase chain reaction (PCR); transcription, including but not limited to reverse transcription; digestion reactions, including enzymatic or chemical digesting agents; or combinations thereof.

V. Exemplary Embodiments

The current teachings, having been described above, may be better understood by reference to examples. The following examples are intended for illustration purposes only, and should not be construed as limiting the scope of the current teachings in any way.

### EXAMPLE 1

### Illustrative Bisulfite Treatment Protocol

A blood sample is obtained from a patient with cervical cancer and the DNA is obtained using BloodPrep™ Chemistry and an ABI Prism® 6700 Nucleic Acid Workstation, according to the manufacturer's protocol (Applied Biosystems). The isolated DNA is bisulfite treated to convert the unmethylated cytosines to uracils, according to the MD Anderson Cancer Center bisulfite treatment protocol (available on the world wide web at mdanderson.org/departments/methylation; MD Anderson Cancer Center—DNA Methylation in Cancer—Protocols—Bisulfite Treatment).

### EXAMPLE 2

### Exemplary Coupled Cleavage and Ligation Reactions

To determine the degree of methylation of a target nucleotide (shown underlined) in a portion of the promoter of the P16 tumor suppressor gene: 5′-CCAGAGGGTGGGGC¹GGACC²GAGTGC³GCTC⁴GGC⁵GGCT-3′ (SEQ ID NO:11) comprising five potentially methylated cytosines (shown with superscript numbers), a cleavage probe set comprising the universal base 5-nitroindole (shown as “N” in Table 1) is synthesized using conventional phosphoramidite chemistries. As shown in Table 1, for each of the probe pairs (i.e., probes 1 and 2; and probes 3 and 4) the upstream cleavage probes comprise the fluorescent reporter group FAM and a sequence complementary to the first target region. The second cleavage probe of each probe pair comprises a sequence that is complementary to the second target region, one of two different flap portions upstream of the sequence complementary to the second target region (shown in brackets), and a polyethylene oxide mobility modifier monomer or dimer (shown as (PEO) and (PEO)₂, respectively). The 5′ ends of the 3′ probes in this exemplary probe set are not phosphorylated.

TABLE 1

Exemplary P16 Cleavage Probe Set.

| | |
|---|---|
| 5′ probes | 3′ probe |

| | |
|---|---|
| FAM- | [GCAGATTG]AATCCNCCCCACCCTCTAA- |
| AACCNCCNAACNCACTCA | (PEO) |
| (SEQ ID NO: 12) | (SEQ ID NO: 13) |
| probe 1 | probe 2 |
| FAM- | [TCTCACCG]GATCCNCCCCACCCTCTAA- |
| AACCNCCNAACNCACTCG | (PEO)₂ |
| (SEQ ID NO: 14) | (SEQ ID NO: 15) |
| probe 3 | probe 4 |

A coupled cleavage-ligation reaction composition is formed comprising the probes of this exemplary P16 cleavage probe set, converted DNA from Example 1, recombinant Pfu FEN-1, and Thermus species AK16D ligase. The reaction is thermocycled to allow the cleavage and ligation reactions to proceed. Two μL of the resulting coupled cleavage-ligation product composition is combined with 18 μL Hi-Di formamide (Applied Biosystems) and the diluted ligation products are separated and detected using capillary electrophoresis in 36 cm capillaries with POP-6™ polymer on the ABI PRISM® 3100 Genetic Analyzer in the gene scan mode using GeneScan® Analysis Software according to the manufacturer's instructions (Applied Biosystems). By comparing the software generated ligation product peak area for the cleavage-ligation product of probes 1 and 2 (“LP1”) with the cleavage-ligation product of probes 3 and 4 (“LP2”) and generating a ratio, the degree to which the exemplary P16 target nucleotide is methylated in the patient is determined. Those in the art will appreciate that one can also determine the degree of target nucleotide methylation by comparing the experimentally obtained LP1 and LP2 peak heights or peak areas with the appropriate standard curve for each. Those in the art will also appreciate that this exemplary coupled cleavage-ligation reaction can be performed in one or a multiplicity of cycles.

Although the disclosed teachings has been described with reference to various applications, methods, and compositions, it will be appreciated that various changes and modifications may be made without departing from the teachings herein. The foregoing examples are provided to better illustrate the disclosed teachings and are not intended to limit the scope of the teachings herein.

What is claimed is:

1. A method for determining the degree of methylation of a target nucleotide comprising,
(a) reacting (1) a target sequence with (2) a first cleavage probe set comprising (i) a first cleavage probe comprising a sequence that is complementary to a first target region and (ii) a second cleavage probe comprising a sequence that is complementary to a second target region and that is downstream from a flap portion, wherein the second target region is located 5′ of the first target region and overlaps the first target region by at least one nucleotide, under effective conditions for the first and second cleavage probes of the first cleavage probe set to anneal to the corresponding first and second target regions, respectively, forming a first hybridization complex;
(b) cleaving the flap portion of the second cleavage probe in the first hybridization complex to generate a cleaved flap and form a second hybridization complex comprising (1) the target sequence, (2) the first cleavage probe, and (3) an annealed fragment of the second cleavage probe having a 5′-terminal nucleotide located adjacent to the 3′-end of the annealed first cleavage probe;
(c) ligating the first cleavage probe to the annealed fragment of the second cleavage probe to generate a first ligation product and form a third hybridization complex comprising the target sequence and the first ligation product; and
(d) determining the degree of methylation of the target nucleotide.

2. The method of claim 1, further comprising:
(e) denaturing the third hybridization complex; and
(f) performing one or more additional cycles of steps (a) through (c) and optionally, step (e).

3. The method of claim 2, further comprising:
(a) combining (1) the first ligation product with (2) a second cleavage probe set comprising (i) a first cleavage probe comprising a sequence that is complementary to a first region of the ligation product and (ii) a second cleavage probe comprising a sequence that is complementary to a second region of the ligation product and that is downstream from a flap portion, wherein the second region of the ligation product is located 5′ of the first region on the ligation product and overlaps the first region of the ligation product by at least one nucleotide, under effective conditions for the first and second cleavage probes of the second cleavage probe set to anneal to the corresponding first and second regions of the ligation product, respectively, and form a fourth hybridization complex;
(b) subjecting the fourth hybridization complex to a cycle of (1) cleaving the flap portion of the second cleavage probe in the fourth hybridization complex to generate a cleaved flap and form a fifth hybridization complex comprising (i) the first ligation product, (ii) the first cleavage probe, and (iii) an annealed fragment of the second cleavage probe having a 5′-terminal nucleotide located adjacent to the 3′-end of the annealed first cleavage probe and (2) ligating the first cleavage probe to the annealed fragment of the second cleavage probe to generate a second ligation product and form a sixth hybridization complex comprising the first ligation product and the second ligation product; and optionally,
(c) denaturing the sixth hybridization complex; and
(d) performing one or more additional cycles of steps (a) and (b), and optionally step (c).

4. The method of claim 2, further comprising:
(a) combining (1) the first ligation product with (2) a first ligation probe set comprising (i) a first ligation probe comprising an upstream first ligation product-binding portion and (ii) a second ligation probe comprising a downstream first ligation product-binding portion, under effective conditions for the first and second ligation probes to anneal to the first ligation product, to forming a seventh hybridization complex comprising the first ligation probe and the second ligation probe of the first ligation probe set and the first ligation product;
(b) ligating the first ligation probe to the second ligation probe to generate a third ligation product and form an eighth hybridization complex comprising the first ligation product and the third ligation product; and
(c) denaturing the eighth hybridization complex.

5. The method of claim 3, further comprising:
(a) combining (1) the second ligation product with (2) a second ligation probe set comprising (i) a first ligation probe comprising an upstream first ligation product-binding portion and (ii) a second ligation probe comprising a downstream first ligation product-binding portion, under effective conditions for the first and second ligation probes of the second ligation probe set to anneal to the second ligation product, forming a ninth hybridization complex comprising the first ligation probe and the second ligation probe of the second ligation probe set and the second ligation product;
(b) ligating the first ligation probe to the second ligation probe to generate a fourth ligation product and form a tenth hybridization complex comprising the second ligation product and the fourth ligation product;
(c) denaturing the tenth hybridization complex; and
(d) performing one or more additional cycles of steps (a) and (b), and optionally step (c).

6. A method for determining the degree of methylation of a target nucleotide comprising,
(a) reacting (1) a target sequence with (2) a first cleavage probe set comprising (i) a first cleavage probe that can hybridize with the target sequence and (ii) a second cleavage probe that (a) can hybridize with the target sequence downstream of the first cleavage probe and (b) contains a flap portion, under effective conditions for the first and second cleavage probes of the first cleavage probe set to hybridize with the target sequence, forming a first hybridization complex;
(b) cleaving the flap portion of the second cleavage probe in the first hybridization complex to generate a cleaved flap and form a second hybridization complex comprising (1) the target sequence, (2) the first cleavage probe, and (3) an annealed fragment of the second cleavage probe having a 5′-terminal nucleotide located adjacent to the 3′-end of the annealed first cleavage probe;
(c) ligating the first cleavage probe to the annealed fragment of the second cleavage probe to generate a first ligation product and form a third hybridization complex comprising the target sequence and the first ligation product;
(d) denaturing the third hybridization complex;
(e) performing one or more additional cycles of steps (a) through (c) and optionally, step (d); and
(f) determining the degree of methylation of the target nucleotide.

7. The method of claim 6, wherein the target sequence is modified using sodium bisulfite.

8. The method of claim 6, wherein a probe of the cleavage probe set comprises a Modification or a degenerate base.

9. The method of claim 8, wherein the Modification comprises a substituted hydrocarbon, a ribonucleotide, an amide bond, a glycosidic bond, an LNA, a nucleotide analog, a universal base, a groove binder, or combinations thereof.

10. The method of claim 6, wherein the ligating comprises a ligase or enzymatically active mutants or variants thereof.

11. The method of claim 6, wherein the second target region overlaps the first target region by more than one nucleotide.

12. The method of claim 6, wherein a probe comprises a reporter group, a hybridization tag, a mobility modifier, an affinity tag, a reporter probe-binding portion, a minor groove binder, or combinations thereof.

13. The method of claim 12, further comprising a reporter probe.

14. The method of claim 13, wherein the determining comprises detecting the reporter group of a ligation product, a ligation product surrogate, a reporter probe or at least part of a reporter probe, or combinations thereof, and evaluating the ligation ratio or threshold cycle during or after a plurality of cycles.

15. The method of claim 6, wherein the determining comprises a mobility-dependent analytical technique, a mass spectrometer, a Substrate, a real-time instrument, or combinations thereof.

16. The method of claim 6, wherein the target sequence, a cleavage probe, a ligation probe, or combinations thereof, is bound to a Substrate.

17. The method of claim 16, wherein the Substrate comprises a hybridization tag complement, a reporter group, an affinity tag, an aptamer, an antibody, or combinations thereof.

18. The method of claim 20, wherein a hybridization complex is formed on a Substrate.

19. The method of claim 18, wherein the target sequence, the cleavage probe, the ligation probe, the hybridization complex, or combinations thereof, are detected on the Substrate.

20. A method for determining the degree of methylation of a target nucleotide, comprising:
a step for interrogating the target nucleotide;
a step for generating a cleaved flap;
a step for generating a ligation product; and
a step for determining the degree of methylation of the target nucleotide.
